(19) United States
(12) Patent Application Publication
(10) Pub. No.: US 2009/0221702 A1
(43) Pub. Date: Sep. 3, 2009

(54) COMPOUNDS

(76) Inventors: Rudiger Woscholski, London (GB); Helen Hailes, London (GB); Macba Numbere, London (GB); Erika Rosivatz, London (GB)

Correspondence Address: ELMORE PATENT LAW GROUP, PC, 515 Groton Road, Unit 1R, Westford, MA, 01886, US

(21) Appl. No.: 11/908,671

(22) PCT Filed: Mar. 17, 2006
(86) PCT No.: PCT/GB06/00961

§ 371 Date: May 22, 2008

(30) Foreign Application Priority Data
Mar. 17, 2005 (GB) 0505509.0

(51) Int. Cl. A61K 31/235; C07C 59/68; A61K 31/192; A61K 31/12; C07C 49/255; C07C 39/16; A61K 31/055; C07C 69/65; A61P 35/00

(52) U.S. Cl. 514/546; 562/465; 514/570; 514/678; 568/308; 568/726; 514/734; 560/219

(57) ABSTRACT

The present invention provides the use of a compound of the Formula: (I) wherein R¹ is C₁₋₅ alkoxy, OCOC₁₋₃Alkyl, O(CH₂)₂O(CH₂)₂O(CH₂)₂OMe, O(CH₂)₂O(CH₂)₂O(CH₂)₂OH or OH; R² is H, (CH₂)ₙOH, OCH₃, Hal or (II) or (III) R³ is H or (CH₂)ₙOH; and R⁴ is C₁₋₆ alkyl, optionally substituted by one or more of Hal, OH, COCH₃, NH₂, NHCH₃, NHMe, NMe₂, OCOCH₃, CO₂H or esters or amides thereof where n is 1-5; and pharmaceutically acceptable salts thereof, in the manufacture of a medicament for use in modulating PKB activity.

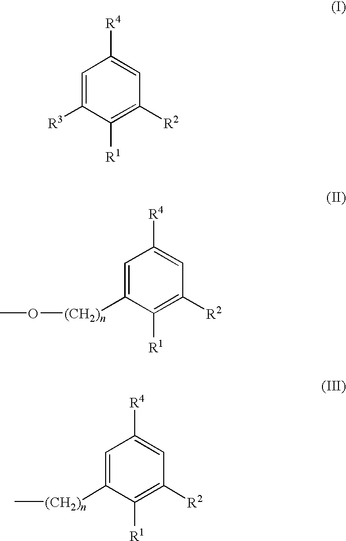

The present invention relates to novel compounds, which are useful as inhibitors and/or activators of protein kinase B (PKB/Akt). As such, these compounds will be useful in the treatment of cancer

Phosphoinositide 3-kinases (PI 3-kinase) are an evolutionary conserved family of enzymes possessing lipid kinase activity who in response to extracellular stimuli are capable of generating a series of 3-phosphorylated phosphoinositide lipids with signalling potential. The resulting cellular effects of PI 3-kinase activity are diverse, including DNA synthesis, chemotaxis, glucose transport and vesicle trafficking. The activation of PI 3-kinases themselves takes place via a number of mechanisms, including receptor tyrosine kinases, Ras and heterotrimeric G-proteins.

One effector of PI 3-kinase responsible for some of the aforementioned effects is protein kinase B (PKB/Akt), a mammalian homologue of the viral oncoprotein v-akt (Staal 1987). PKB is recruited to the plasma membrane in response to growth factor stimulation via the binding of 3-phosphoinositides to its PH domain which facilitates its phosphorylation at two distinct sites and subsequent activation. The first phosphorylation site, threonine-308 (T308) lies in the activation loop of PB and is phosphorylated by phosphoinositide-dependent kinase-1 (PDK-1). The second site, serine-473 (S473) lies in the C-terminal hydrophobic regulatory domain, and is phosphorylated by an as yet unidentified kinase (Chang, Lee et al. 2003). To date several S473 candidate kinases have been postulated, including PDK-1, mitogen-activated protein kinase-activated protein kinase 2, intergrin-linked kinase (ILK) and PKB itself (Brazil, Park et al. 2002; Hill, Feng et al. 2002). It remains to be seen whether any of these kinases or a so far unidentified kinase is responsible for the phosphorylation of this particular site. Other protein kinases of the AGC kinase family such as protein kinase C delta (PKCδ) and p70^{S6K} share a similar activation mechanism via the phosphorylation of their homologous residues (Newton 2003). The activation of all the aforementioned kinases is susceptible to PI 3-kinase inhibition by LY294002 and wortmannin. Effectors of PKB include Bad, GSK-3 (glycogen synthase kinase-3) and mTOR (mammalian target of rapamycin) (Vivanco and Sawyers 2002). mTOR is a regulator of protein synthesis and is instrumental in PKCδ activation (Parekh, Ziegler et al. 2000). Like PI 3-kinase, studies of mTOR signalling have been aided by the use of pharmacological agents. mTOR activity is inhibited by rapamycin, via its binding to FKBP12, thus inhibiting events distal to mTOR (Sabers, Martin et al. 1995).

Thus, Phosphoinositide signalling is a key element in controlling cell death, survival and fate. In particular, cell survival is an important mechanism of the natural defence against cancer. Cell survival is controlled by phosphoinositide 3-kinase products, which in turn activate a particular protein kinase, called PKB or Akt. PKB/Akt is phosphorylated by other kinases subsequently leading towards full activation of its own catalytic abilities and thus progressing the cell survival signal through this protein kinase cascade. Unravelling the elements in control of PKB phosphorylation has been the focus of many research groups and drug development teams.

We have now identified compounds which are capable of inhibiting and/or activating PKB.

Thus, in a first aspect, the present invention provides the use of a compound of the formula:

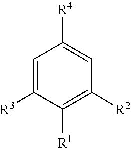

wherein R¹ is C₁₋₅ alkoxy, OCOC₁₋₃Alkyl, O(CH₂)₂O(CH₂)₂O(CH₂)₂OMe, O(CH₂)₂O(CH₂)₂O(CH₂)₂OH or OH;
R² is H, (CH₂)ₙOH, OCH₃, Hal or

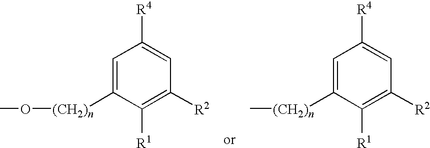

R³ is H or (CH₂)ₙOH; and
R⁴ is C₁₋₆ alkyl, optionally substituted by one or more of Hal, OH, COCH₃, NH₂, NHCH₃, NHMe, NMe₂, OCOCH₃, CO₂H or esters or amides thereof
where n is 1-5;
and pharmaceutically acceptable salts thereof, in the manufacture of a medicament for use in modulating PKB activity.

In the context of the present invention, halogen means F, Cl, I or Br, preferably Cl, I or Br.

For the purposes of this invention, alkyl relates to both straight chain and branched alkyl radicals of 1 to 6 carbon atoms including but not limited to methyl, ethyl, n-propyl, isopropyl, n-butyl, sec-butyl, isobutyl, tert-butyl n-pentyl, n-hexyl. In particular, alkyl relates to a group having 1, 2, 3, 4, 5 or 6 carbon atoms. The term alkyl also encompasses cycloalkyl radicals including but not limited to cyclopropyl, cyclobutyl, CH₂-cyclopropyl, CH₂-cyclobutyl, cyclopentyl or cyclohexyl. In particular, cycloalkyl relates to a group having 3, 4, 5 or 6 carbon atoms. Cycloalkyl groups may be optionally substituted or fused to one or more carbocyclyl or heterocyclyl group.

As discussed herein, the compounds of the present invention find use as inhibitors and/or activators of PKB, and thus as agents for use in the treatment of cancer. In particular, the compounds described herein find use in cancers where up regulation of PKB is implicated and more particularly where up-regulation together with mutation of PTEN is implicated. Thus, cancers such as ovarian, breast, prostrate, thyroid and pancreatic cancers are particular targets of the compounds.

Those compounds described herein as activators find use in preventing cell death. Thus, they find use in treating degenerative disorders degenerative diseases of those tissues that are unable to reproduce, i.e. neurons (Alzheimer, stroke, etc) or heart (infarct, hypoxia) and skeletal muscle (sports injuries) tissue, respectively (Glass 2003; Matsui, Nagoshi et al. 2003; Tatton, Chen et al. 2003).

Thus, in a second aspect the present invention provides a pharmaceutical formulation comprising one or compounds as defined herein, optionally together with one or more pharmaceutically acceptable diluents, carriers and/or excipients.

The compositions of the invention may be presented in unit dose forms containing a predetermined amount of each active ingredient per dose. Such a unit may be adapted to provide 5-100 mg/day of the compound, preferably either 5-15 mg/day, 10-30 mg/day, 25-50 mg/day, 40-80 mg/day or 60-100 mg/day. For compounds of formula I, doses in the range 100-1000 mg/day are provided, preferably either 100-400 mg/day, 300-600 mg/day or 500-1000 mg/day. Such doses can be provided in a single dose or as a number of discrete doses. The ultimate dose will of course depend on the condition being treated, the route of administration and the age, weight and condition of the patient and will be at the doctor's discretion.

The subject of the present invention is most preferably administered in the form of appropriate compositions. As appropriate compositions there may be cited all compositions usually employed for systemically or locally administering drugs. The pharmaceutically acceptable carrier should be substantially inert, so as not to act with the active component. Suitable inert carriers include water, alcohol, polyethylene glycol, mineral oil or petroleum gel, propylene glycol and the like. Said pharmaceutical preparations may be formulated for administration in any convenient way for use in human or veterinary medicine.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject agents may be simply dissolved or suspended in sterile water. In certain embodiments, the pharmaceutical preparation is non-pyrogenic, i.e., does not elevate the body temperature of a patient. The phrase “effective amount” as used herein means that amount of one or more agent, material, or composition comprising one or more agents of the present invention which is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the patient, the route of administration, etc. A skilled medical practitioner can readily determine the appropriate dose using methods well known in the medical arts. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, one or more agents may contain a basic functional group, such as amino or alkylamino, and are, thus, capable of forming pharmaceutically acceptable salts with pharmaceutically acceptable acids.

The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (Berge, Bighley et al. 1977). The pharmaceutically acceptable salts of the agents include the conventional non-toxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, the one or more agents may contain one or more acidic functional groups and, thus, are capable of forming pharmaceutically acceptable salts with pharmaceutically acceptable bases. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine.

Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra). Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and ublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent. Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste. In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, olyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin apsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable non-irritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the agents. Formulations of the present invention which are suitable for vaginal administration also include pessaries, tampons, creams, gels, pastes, foams or spray formulations containing such carriers as are known in the art to be appropriate. Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle. Injectable depot forms are made by forming microencapsule matrices of the subject compounds in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions which are compatible with body tissue.

When the compounds described herein are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier. Apart from the above-described compositions, use may be made of covers, e.g., plasters, bandages, dressings, gauze pads and the like, containing an appropriate amount of a therapeutic. As described in detail above, therapeutic compositions may be administered/delivered on stents, devices, prosthetics, and implants.

In a third aspect the present invention provides a compound as defined herein for use in medicine, particularly in the treatment of cancer.

The compounds described herein are available from commercial sources or are readily synthesised using standard chemical methodologies and common general knowledge.

In addition, the following compounds are novel and form further aspects of the invention:

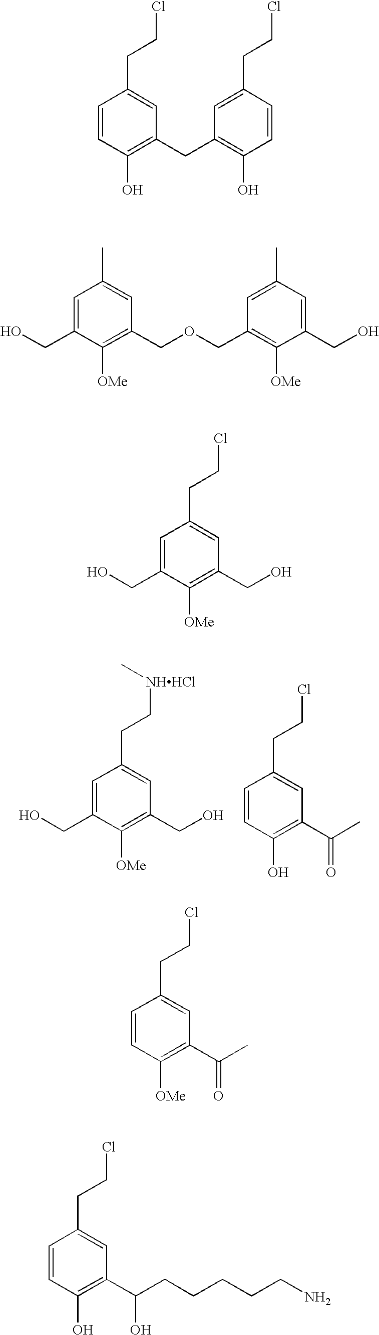

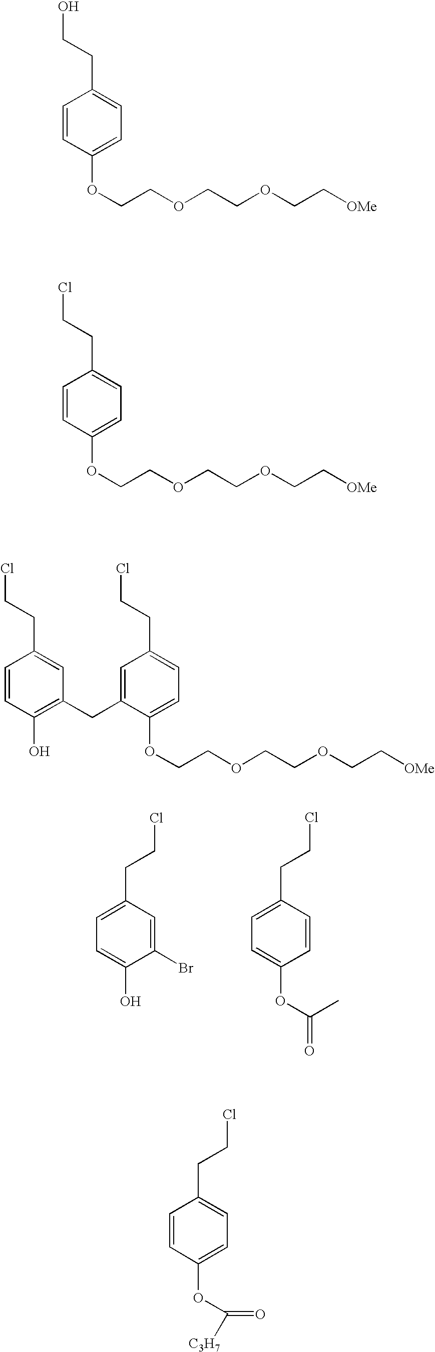

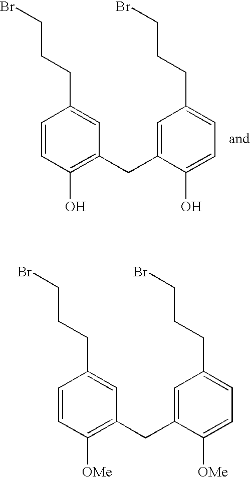

Finally, the present invention provides a compound of the formula:

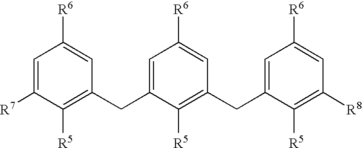

wherein R⁵ is C₁₋₅ alkoxy or OH;
R⁶ is C₁₋₅ alkyl, optionally substituted by Hal, NHCH₃, CO₂H or esters or amides thereof; and
R⁷ and R⁸ are independently (CH₂)_{q}OH where q is 2-5;
and pharmaceutically acceptable salts thereof. The use of such compounds in the manufacture of a medicament for use in modulating PKB activity is also provided.

The invention will now be described with reference to the following examples, which should in no way be construed as limiting the scope of the invention. Preferred features of each aspect of the invention are as for each other aspect, mutatis mutandis.

The examples refer to the figures, in which:

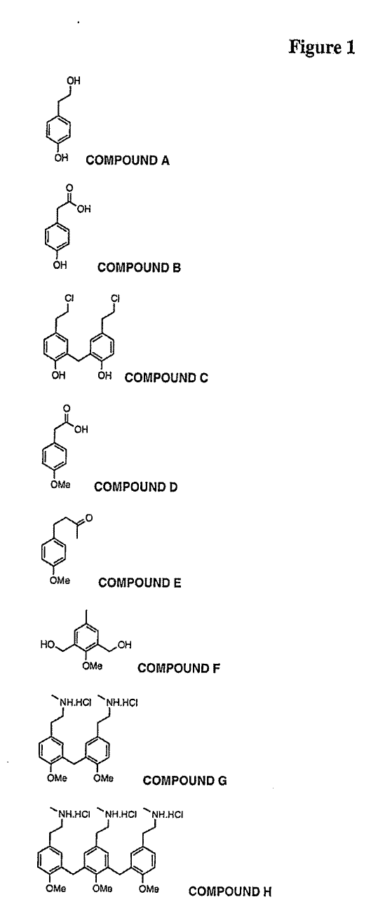

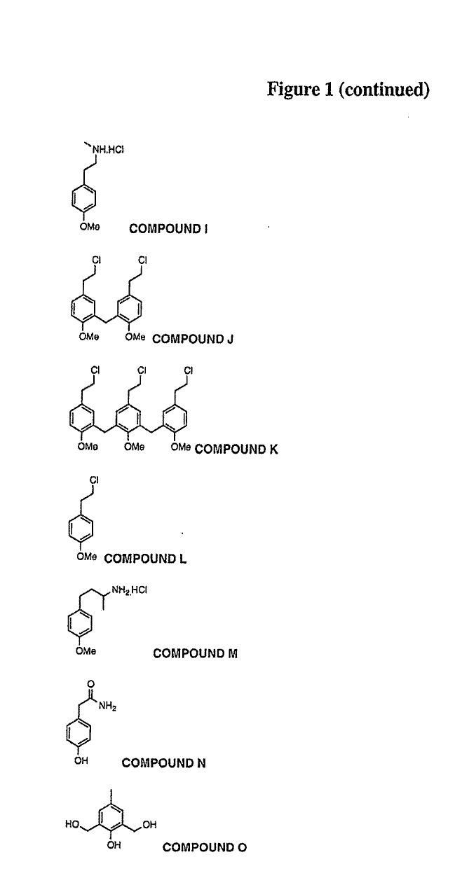

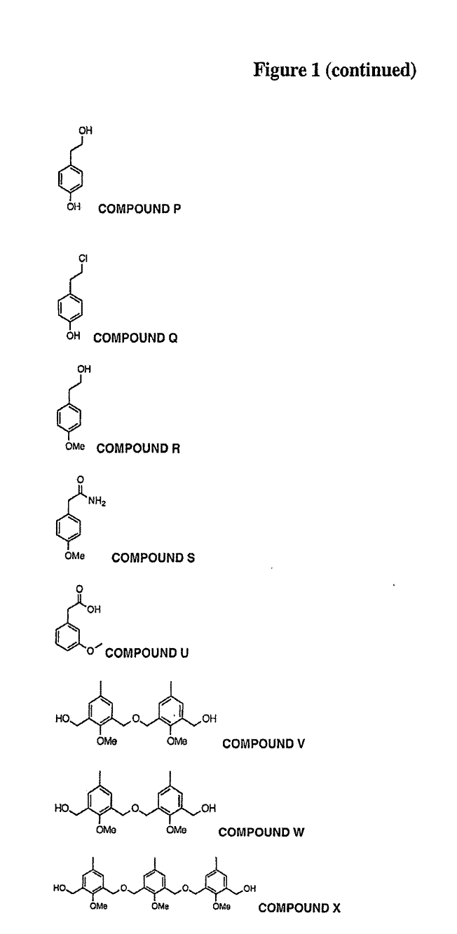

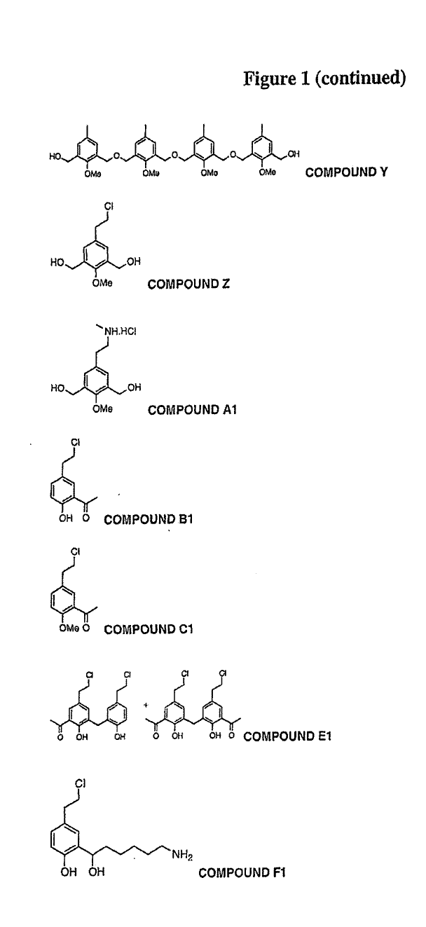

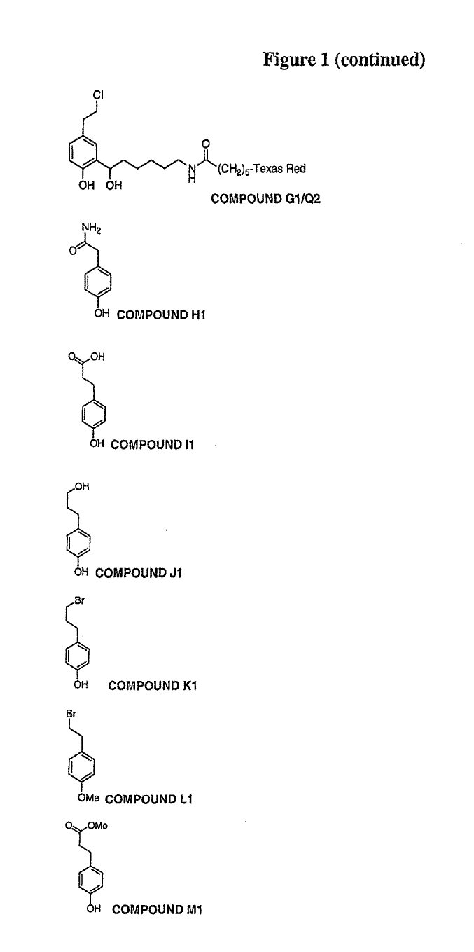

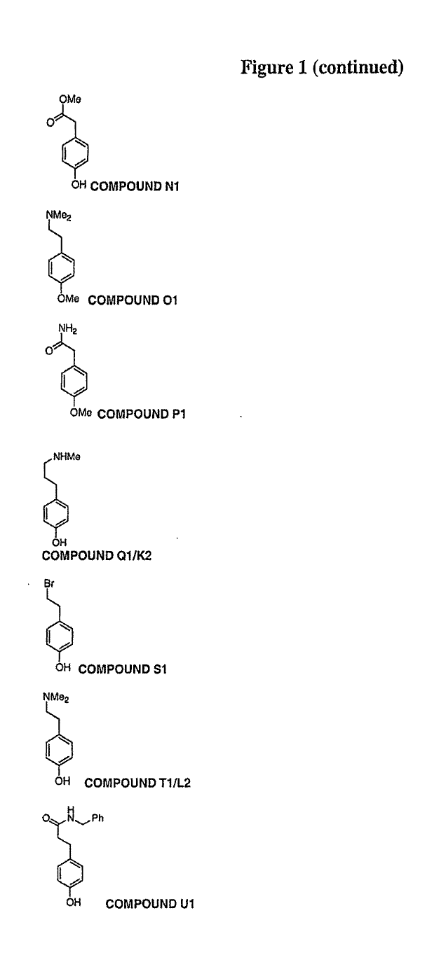

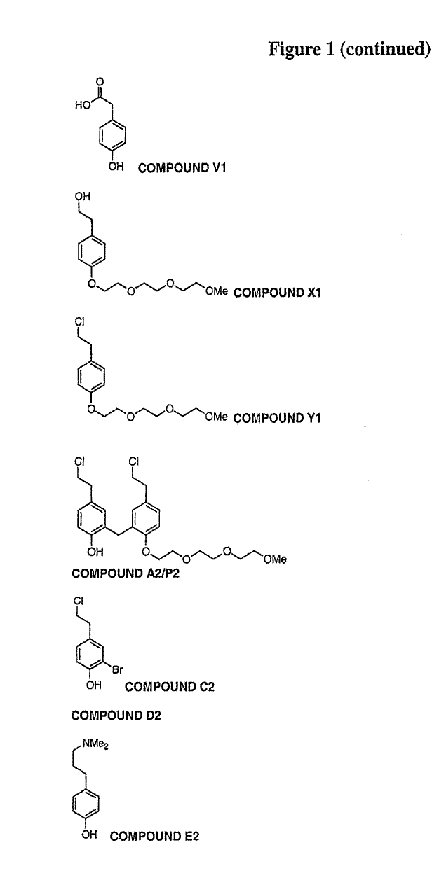

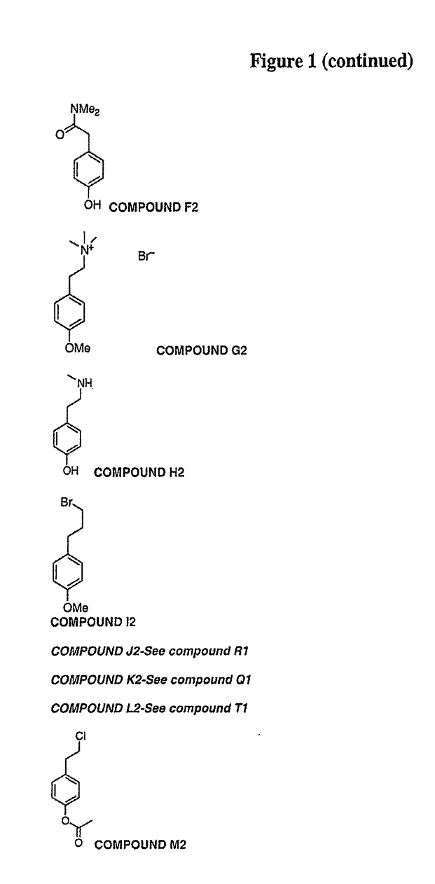

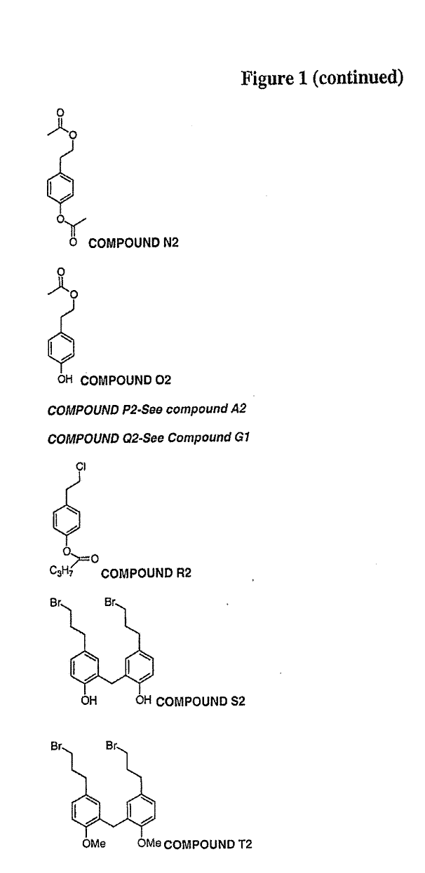

FIG. 1 provides compounds of the invention;

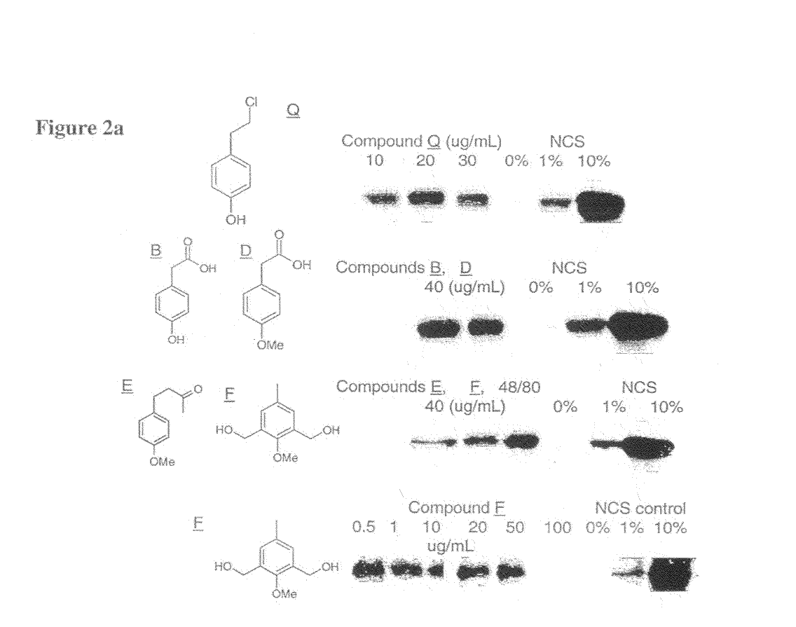

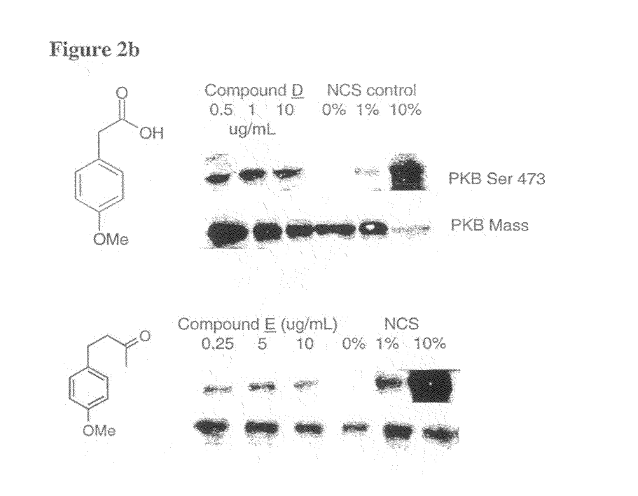

FIG. 2 provides the results of western blots, illustrating phosphorylation of PKB when treated with various compounds of the invention;

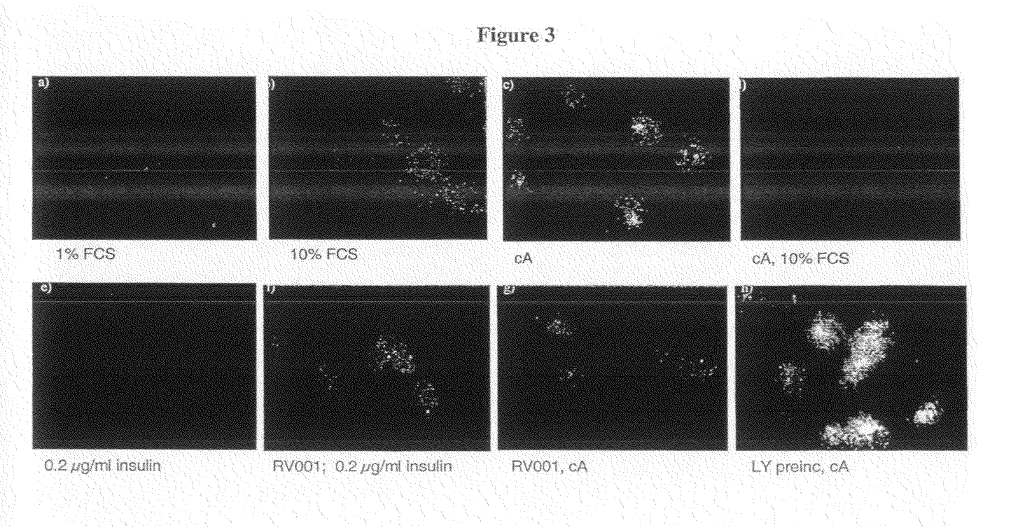

FIG. 3 illustrates that compound Q of the invention induces phosphorylation of PKB. Overlay of green and blue fluorescence channels where phosphorylation of PKB on S473 is indicated by the increase of green staining with a FITC labelled phospho-specific antibody and dapi stained nuclei are shown in false colour red. Upper panel: where indicated starved Cos6 cells were treated with serum and/or 15 μg/ml cQ for 10 min. Lower panel: e) starved Cos6 cells were treated with non-stimulatory concentrations (0.2 μg/ml) of insulin. f) pretreatment with 500 nM PTEN inhibitor RV001 before insulin challenge was sufficient to induce phosphorylation of PKB, whereas g) 15 μg/ml cQ inhibited PI(3,4,5)P₃ mediated PKB activation. h) 30 min preincubation with 100 μM of PI3-kinase inhibitor LY29400 increased cQ induced PKB phosphorylation (compared to c);

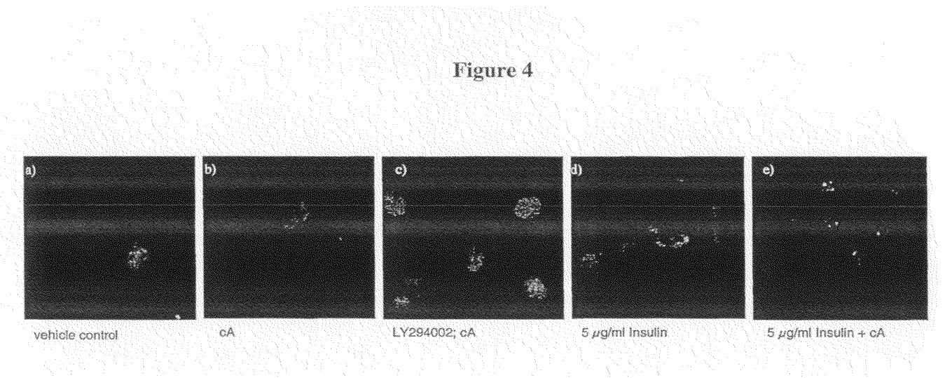

FIG. 4 illustrates that compound Q inhibits insulin-stimulated actin remodelling. Overlay of red (F-actin) and blue (nuclei) fluorescence channels. Where indicated, starved Cos6 cells were stimulated with 15 μg/ml cQ and/or 5 μg/ml insulin for 10 min. c) Cells were preincubated with 100 μM LY294002 for 30 min, before stimulation. Rhodamine-labelled phalloidin staining demonstrated that a) starved Cos6 fibroblasts form stress fibers which are remodeled after d) insulin stimulation into polymerised F-actin juxtaposed to the plasma membrane. b) cA also induces the loss of stress fibers in starved fibroblasts. But unlike insulin, cQ is capable of reorganising the cytoskeleton c) independent of PI3-kinase and e) interferes with insulin-stimulated stress fiber breakdown; and

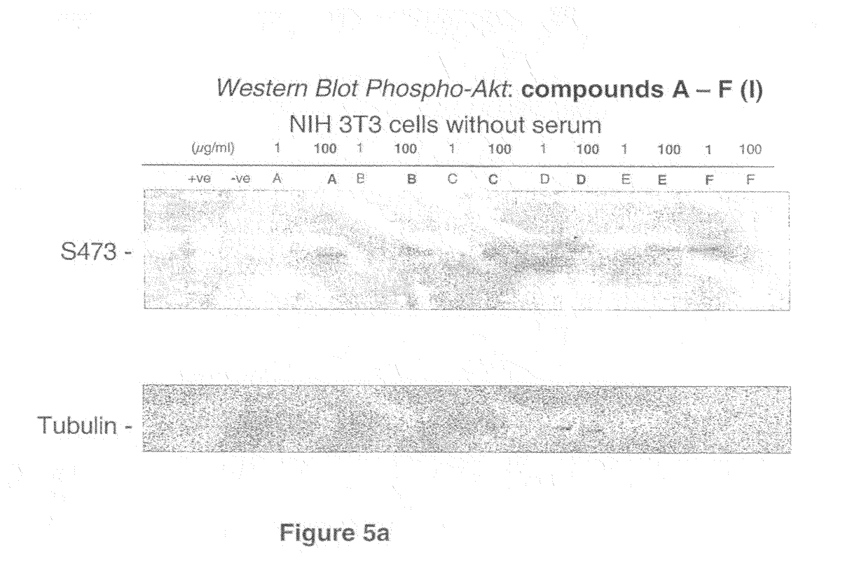

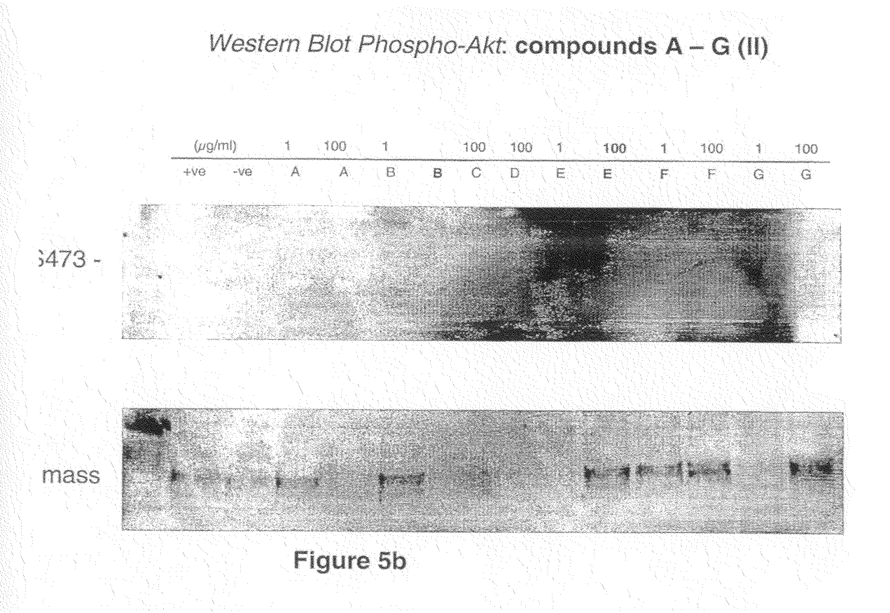

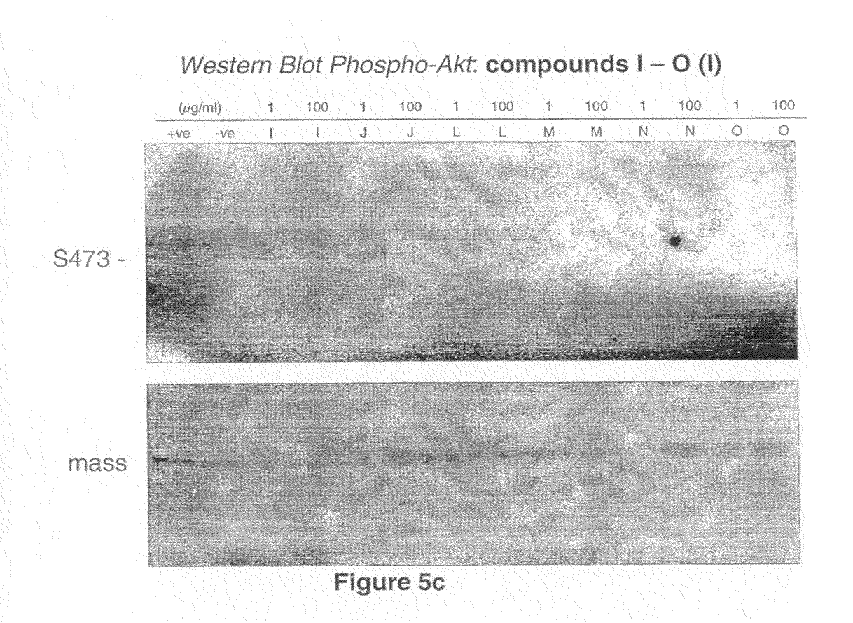

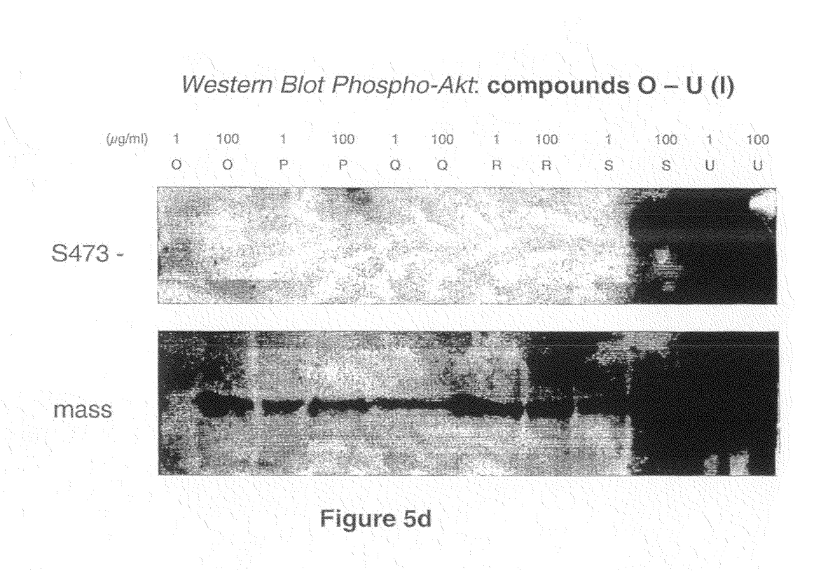

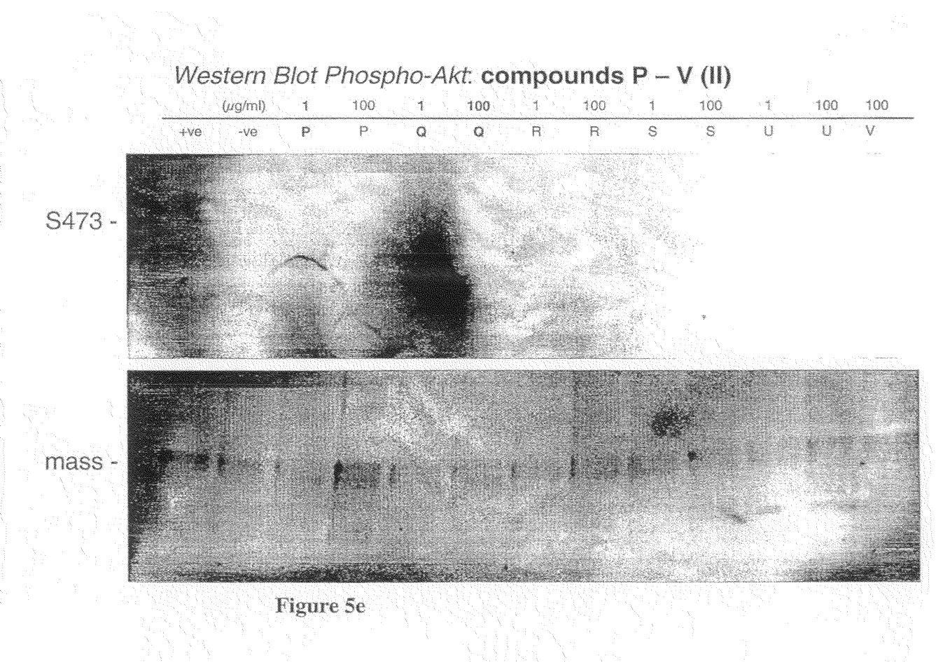

FIG. 5 provides the results of further western blots, illustrating activation of PKB with various compounds of the invention.

### EXPERIMENTAL

### Synthesis of Various Compounds of the Invention

Starting materials were obtained from commercial suppliers and used without further purification unless otherwise stated. Anhydrous solvents were HPLC grade. All non-aqueous reactions were carried under an atmosphere of nitrogen, using oven or flame-dried glassware. Water refers to deionised water, and brine to saturated sodium chloride solution. Solvents were removed under reduced pressure using a Büchi rotary evaporator. Flash column chromatography was carried out using silica gel (35-70 μm particles). Thin layer chromatography was performed using on commercially available pre-coated aluminium plates. Visualisation of plates was performed by fluorescence quenching, or staining with KMnO₄ or phosphomolybdic acid.

¹H and ¹³C NMR spectra were recorded on a Bruker Avance AMX-300 Fourier Transform spectrometer. Chemical shift values are quoted in parts per million (ppm) downfield of tetramethylsilane, and values of coupling constants (J) are given in values of Hz. NMR spectra were recorded at 300 K, unless otherwise stated.

Infrared spectra were recorded using a Shamadazu FTIR-8700 infrared spectrophotometer. Melting points were determined on a Gallenkamp melting point apparatus and are uncorrected. Mass spectra were recorded using a Micromass LCT-KA111 electrospray mass spectrometer. Accurate molecular weights were carried out by staff at the Department of Chemistry, University College London.

### 3,3′-Methylenebis[1-(2-chloroethyl)-4-hydroxybenzene]

### Compound C

All chemicals and reagents were purchased from commercial suppliers—Sigma Aldrich Company Ltd, Avocado Research Chemicals Ltd and Lancaster Synthesis without further purification. Solvents were used directly without further purification unless otherwise indicated. The water used for washings was deionised. Brine refers to saturated aqueous sodium chloride. ¹H NMR and ¹³C NMR spectroscopy were carried out using a Bruker instrument AMX at 300 MHz and 75 MHz respectively. IR spectra were obtained on a Nicolet FT-IR machine. Melting points were determined using Gallenkamp melting point apparatus.

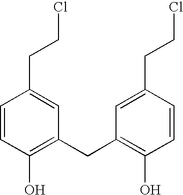

Sulfuric acid (25% w/w in water; 50 ml, 142.7 mmol) and formaldehyde (37% w/v in water; 1.2 ml, 14.8 mmol) were added to 4-hydroxyphenethyl chloride (1.96 ml, 12.5 mmol). The reaction was stirred using a mechanical stirrer and heated at reflux for 2 h at 70° C. After cooling, the aqueous layer was decanted off and the residual white solid was dissolved in ethyl acetate (60 ml). The organic layer was washed with water (2×30 ml) and brine (2×30 ml). The organic layer was dried (magnesium sulfate) and evaporated in vacuo to give a solid. The product was purified by recrystallisation from chloroform to give the title compound as a white powder (49%, 0.987 g).

mp 161-165° C. (chloroform);

IR (nujol)/cm⁻¹ 1591 m, 1608 m, 2932 w, 3020 w, 3225 s (O—H);

δ_{H}(300 MHz; CDCl₃) 3.01 (4H, t, J 7.4 Hz, 2-H), 3.72 (4H, t, J 7.4 Hz 1-H), 3.93 (2H, s, CH₂), 6.36 (2H, br, OH), 6.81 (2H, d, J 8.2 Hz, 5-H), 7.00 (2H, dd, J 8.2 and 2.2 Hz, 6-H), 7.15 (2H, d, J 2.2 Hz, 2-H);

δ_{C}(75 MHz; CDCl₃) 32.5 (2H, ArCH₂Ar), 38.5 (CH₂CH₂Cl,), 45.4 (CH₂CH₂Cl), 116.3, 126.8, 128.5, 131.2, 131.3, 151.7;

m/z (FAB) 324 (M⁺, 52%), 307 ([M-OH]⁺, 24), 289 ([M-OH—OH₂]⁺, 24).

m/z HRMS calcd for C₁₇H₁₈C₁₂O₂ [M]⁺324.06838. found 324.06843.

4-(4-Methoxyphenyl)-2-aminobutane hydrochloride—Compound M (MGN-M253) (S. K. Chattopadhyay, K. V. Sashidhara, V. Koneni, V. Tripathi, A. K. Tripathi, V. Prajapati, S. Kumar, U. S. (2001) 6252114)

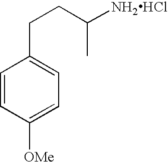

A mixture of 4-(4-methoxyphenyl)-2-butanone (2.09 g, 11.74 mmol), ammonium acetate (9.06 g, 11.75 mmol) and sodium cyanoborohydride (0.52 g, 0.82 mmol) in methanol (30 ml) was stirred at room temperature for 72 h. The reaction mixture was acidified with concentrated HCl (10 ml) and the solvent remove in vacuo. Water was then added and the unreacted starting material was extracted using diethyl ether (3×50 ml). The aqueous solution was made basic using potassium hydroxide pellets, saturated with sodium chloride (2 g) and extracted with diethyl ether (3×100 ml). The combined organic extracts were dried with magnesium sulfate and evaporated in vacuo to give 3-(4-methoxyphenyl)-1-methylpropylamine as a colourless viscous liquid. The hydrochloride was then prepared by adding 20% HCl-methanol (3 ml) to the amine. The mixture was evaporated to dryness and recrystallised from dichloromethane to give 3-(4-methoxyphenyl)-1-methylpropylamine hydrochloride as a white solid (0.31 g, 68%).

νₘₐₓ(film)/cm⁻¹ 1514, 1612, 2937, 2997, 3423 (N—H stretch).

¹H NMR (300 MHz; CDCl₃) 1.52 (3H, d, J 6.6 Hz), 2.09 (2H, m), 2.88 (2H, m), 3.66 (1H, m), 4.01 (3H, s), 7.17 (2H, d, J 8.7 Hz), 7.45 (2H, d, J 8.7 Hz);

¹³C NMR (75 MHz; CDCl₃) 17.7, 30.1, 36.0, 47.6, 55.7, 114.5, 129.8, 133.9, 157.5;

m/z (ES+) 180 ([M—Cl]⁺, 100%);

m/z HRMS calcd for C₁₁H₁₈NO [M+H]⁺ 180.13883. found 180.13863.

### 2,6-Bis(hydroxymethyl)-4-anisole—Compound F and for Use in Synthesis of Compound V

(See B. Masci, S. Saccheo, Tetrahedron, 1993, 49, 10739 for synthesis.)

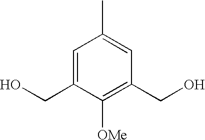

mp 98-100° C. (chloroform) (lit, 103-104° C.)

νₘₐₓ (film)/cm⁻¹ 1475 w, 2835 w, 2850 w, 2910 w, 2935 w, 3180 br, 3290 br (O—H stretch);

¹H NMR (300 MHz; CDCl₃) 2.32 (3H, s, 1-H), 3.84 (3H, s, 7-H), 4.70 (4H, s, 6-H), 7.13 (2H, s, 3-H);

¹³C NMR (75 MHz; CDCl₃) 20.9 (CH₃), 61.2, 62.4, 129.7, 133.8, 134.5, 154.2;

m/z (ES⁺) 182 (M⁺, 100%), 165 ([M-CH₃]⁺, 98).

### [3-(3-hydroxymethyl-2-methoxy-5-methylbenzyloxymethyl)-2-methoxy-5-methylphenyl]-methanol—Compound V (MGN-V481(di))

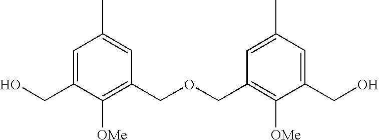

Step 1: To a suspension of wang (polymer-bound p-benzyloxybenzyl alcohol) resin (3.07 g, 4.5 mmol) in dry dichloromethane (30 ml), was added trichloroacetonitrile (4.5 ml, 44.88 mmol). The mixture was cooled to 0° C., 1,8-diazabicyclo[5.4.0]undec-7-ene (DBU) (0.3 ml, 2.00 mmol) was added dropwise and the reaction mixture was shaken for 1 h at 0° C. The resin was collected in a sintered glass funnel and washed sequentially with dichloromethane, tetrahydrofuran, tetrahydrofuran/methanol (1:1), methanol, tetrahydrofuran/methanol (1:1), tetrahydrofuran and dichloromethane.

Step 2: The resulting resin (0.83 g, 1.22 mmol) was washed with tetrahydrofuran (2×3 ml) under nitrogen and then suspended in dry tetrahydrofuran (20 ml). The alcohol, 2,6-bis(hydroxymethyl)-4-anisole (3.30 g, 18.13 mmol) was added and the reaction mixture was shaken for 20 mins. Then boron-triflouride dietherate (0.093 ml, 0.76 mmol) was added dropwise and the resin suspension was left shaking at room temperature for 18 h. The resin was collected in a sintered glass funnel and washed sequentially with tetrahydrofuran, tetrahydrofuran/methanol (1:1), methanol, tetrahydrofuran/methanol (1:1), tetrahydrofuran and dichloromethane.

Step 3: To a suspension of the resin in dry dichloromethane the resin was washed as before. The resin (0.17 g, 0.25 mmol) was re-suspended in a solution of 1% trifluoroacetic acid/dichloromethane (5.0 ml). The resin suspension was shaken for 4 h and then washed sequentially with dichloromethane, tetrahydrofuran, tetrahydrofuran/methanol (1:1), methanol, tetrahydrofuran/methanol (1:1), tetrahydrofuran and dichloromethane. The organic layer was washed with water (2×50 ml) and brine (2×40 ml), dried with magnesium sulfate and reduced in vacuo. The product was purified by HPLC to afford [3-(3-hydroxymethyl-2-methoxy-5-methyl-benzyloxymethyl)-2-methoxy-5-methyl-phenyl]-methanol (MGN-V481(di)) (86%).

νₘₐₓ(film)/cm⁻¹ 1385 m, 1614 m, 1682 s, 1714 m, 2928 w, 2964 w, 3418 b (O—H stretch);

¹H NMR (300 MHz; CDCl₃) 2.32 (6H, s), 3.80 (6H, s), 4.62 (4H, s), 4.71 (4H, s), 7.13 (2H, s), 7.21 (2H, s);

¹³C NMR (75 MHz; CDCl₃) 20.8, 61.4, 62.4, 67.4, 129.5, 130.4, 131.0, 132.5, 134.0, 154.3;

m/z (ES+) 369 (M+Na, 100%);

m/z HRMS calcd for C₂₀H₂₆O₅ [M+Na] 369.16725. found 369.16726.

### [5-(2-Chloroethyl)-3-hydroxymethyl-2-methoxyphenyl]-methanol

### Compound Z (MGN-Z594)

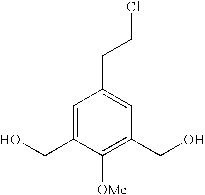

To a solution of 4-methoxyphenethyl chloride (5 ml) in dichloromethane (40 ml), aluminium chloride (9.67 g, 72.52 mmol) was added. The mixture was cooled in ice and then acetyl chloride (5.16 ml, 72.46 mmol) was added dropwise. The reaction mixture was heated at reflux at 55° C. for 18 h. Once cooled the reaction mixture was poured into ice cautiously and the product was extracted using dichloromethane (3×80 ml). The organic layer was washed with brine (2×100 ml), dried with magnesium sulfate, concentrated in vacuo and purified by flash chromatography (eluent: diethyl ether/hexane (1:3)) affording the bis-acetylated intermediate (4.05 g, 64%).

To a solution of the intermediate (2.58 g, 10.73 mmol) and potassium carbonate (3.00 g, 21.72 mmol) in acetone (30 ml), was added iodomethane (1.34 g, 21.44 mmol). The reaction was heated at reflux for 18 h. Once cooled the potassium carbonate was filtered off washing thoroughly with copious amounts of acetone (3×100 ml) and the acetone was then removed in vacuo. The product was redissolved in diethyl ether (60 ml) and washed with water (2×40 ml) and brine (2×40 ml). The product was concentrated in vacuo to afford the O-methoxy bis-acetylated intermediate (2.13 g, 78%).

Anhydrous methanol (7 ml) was added to the O-methoxy intermediate (0.65 g, 2.55 mmol) under nitrogen and then cooled to 0° C. in ice. 13% Sodium hypochlorite (24 ml, 20.55 mmol) was added dropwise and the mixture was left to stir at room temperature for 18 h. Then 18.5% aqueous hydrochloric acid (5 ml) was added slowly and the mixture was left to stir at room temperature for 3 h. The reaction mixture was then concentrated in vacuo and redissolved in anhydrous methanol (10 ml) under nitrogen. The mixture was cooled in ice and thionyl chloride (056 ml, 7.64 mmol) was added dropwise. The mixture was then left to stir for 18 h then the solvent and excess thionyl chloride were removed in vacuo and water (10 ml) was added. The intermediate was extracted using dichloromethane (3×15 ml), washed with brine (2×20 ml)), dried with magnesium sulfate and concentrated in vacuo. To a mixture of lithium aluminium hydride (0.17 g, 4.52 mmol) in tetrahydrofuran (8 ml) under nitrogen (stirred for 30 mins beforehand), was added dropwise the intermediate (0.41 g, 1.42 mmol) in tetrahydrofuran (4 ml). The reaction was stirred at room temperature for 4 h. Water (2 ml), followed by 2M sodium hydroxide (1.5 ml) and then water (2 ml) again was added cautiously. The product was extracted with dichloromethane (3×30 ml) to afford [5-(2-chloro-ethyl)-3-hydroxymethyl-2-methoxy-phenyl]-methanol as a pale yellow viscous liquid (0.25 g, 57%) which was purified by flash chromatography (eluent: dichloromethane/methanol, 10:1).

¹H NMR (300 MHz; CDCl₃) 3.04 (2H, t, J 7.3 Hz), 3.66 (2H, t, J 7.3 Hz), 3.85 (3H, s), 4.73 (4H, s), 7.20 (2H, s);

¹³C NMR (75 MHz; CDCl₃) 38.7, 45.0, 61.1, 62.3, 129.3, 134.2, 134.7, 155.1;

m/z (ES+) 253 (M+Na, 100%), 219 ([(M-OH₂)+Na]⁺, 50);

m/z HRMS calcd for C₁₁H₁₅ClO₃ [M+Na] 253.06019. found 253.06017.

### [3-Hydroxymethyl-2-methoxy-5-(2-methylamino-ethyl)-phenyl]-methanol hydrochloride—Compound A1 (MGN-A1598)

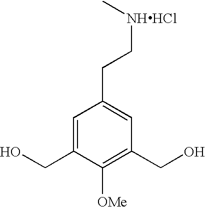

To a solution of compound Z (72 mg, 0.27 mmol), was added 33% methylamine in ethanol (2 ml, 13.37 mmol) and the reaction was stirred for 10 days at rt. The solvent was removed in vacuo and water was added to the reaction mixture followed by 1M hydrochloric acid (1 ml). The organic layer was extracted using diethyl ether. The aqueous layer was made basic with 2M potassium hydroxide (0.5 ml) and the product was concentrated in vacuo to give an orange liquid. 18.5% Hydrochloric acid/methanol (0.1 ml) was added to the amine product and left to stir for 30 mins. The solution was reduced in vacuo and the product separated between water (30 ml) and dichloromethane (2×20 ml). The aqueous layer was reduced in vacuo to afford compound A1, 3-hydroxymethyl-2-methoxy-5-(2-methylaminoethyl)-phenyl]-methanol hydrochloride (63 mg, 89%).

νₘₐₓ(film)/cm⁻¹ 1477 s, 1633 w, 1649 w, 1710 w, 2885 w, 2962 w, 3362 br (N—H stretch);

¹H NMR (300 MHz; CDCl₃) 2.61 (3H, s), 2.92 (2H, t, J 7.5 Hz), 3.13 (2H, t, J 7.5 Hz), 3.71 (3H, s), 4.60 (4H, s), 7.22 (2H, s);

¹³C NMR (75 MHz; CDCl₃) 31.5 (C-1), 33.2 (C-4), 50.4 (C-3), 58.9 (C-9), 62.9 (C-10), 130.0 (C-6), 133.4 (C-7), 134.3 (C-5), 154.7 (C-8);

m/z (ES+) 226 [M-Cl]⁺, 100%);

m/z HRMS calcd for C₁₂H₂₀ClNO₃ [M-Cl]⁺226.14377. found 226.14381.

### 1-[5-(2-Chloroethyl)-2-hydroxyphenyl]-ethanone—Compound B1 (MGN-B1558F1) and 1-[5-(2-Chloroethyl)-2-methoxyphenyl]-ethanone Compound C1 (MGN-C1557F2)

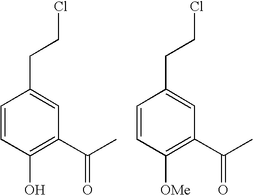

Aluminium chloride (764 mg, 5.73 mmol) was added to solution of 4-methoxyphenethyl chloride (0.4 ml, 2.64 mmol) in dichloromethane under nitrogen. The mixture was cooled in ice to 0° C. and then acetyl chloride (0.41 ml, 5.76 mmol) was added dropwise. The reaction mixture was left to stir at room temperature for 24 h. The mixture was cautiously poured into ice and the organic layer was extracted using dichloromethane (3×30 ml). The combined organic extracts was washed with brine, dried with magnesium sulfate and concentrated in vacuo. The product was purified via flash chromatography (eluent: diethyl ether/hexane, 1:4) yielding 1-[5-(2-chloro-ethyl)-2-hydroxy-phenyl]-ethanone (MGN-B1558F1) (24 mg, 5%) and 1-[5-(2-chloro-ethyl)-2-methoxy-phenyl]-ethanone (MGN-C1557F2) (11 mg, 2%) as bi-products from the reaction to generate the bis-acylated product.

### B1

νₘₐₓ(film)/cm⁻¹ 1487 s, 1620 m, 1643 s, 1650 s, 2959 m, 3011 w;

¹H NMR (300 MHz; CDCl₃) 2.63 (3H, s), 3.03 (2H, t, J 7.1 Hz), 3.70 (2H, t, J 7.1 Hz), 6.95 (1H, d, J 8.5 Hz), 7.34 (1H, J 8.5 Hz), 7.58 (1H, s);

¹³C NMR (75 MHz; CDCl₃) 26.8, 39.2, 45.2, 118.8, 119.9, 128.6, 130.9, 137.1, 161.5, 204.5;

m/z (ES+) 199 ([M+H]⁺, 100%);

m/z HRMS calcd for C₁₀H₁₁ClO₂ [M+H]⁺ 199.05258. found 199.05115.

### C1

¹H NMR (300 MHz; CDCl₃) 2.61 (3H, s, 11-H), 3.02 (2H, t, J 7.2, 2-H), 3.69 (2H, t, J 7.2 Hz, 1-H), 3.90 (3H, s, 9-H), 6.93 (1H, d, J 8.4 Hz, 6-H), 7.33 (1H, d, J 8.4 Hz, 4-H), 7.56 (1H, s, 5-H);

¹³C NMR (75 MHz; CDCl₃) 32.0, 38.1, 45.1, 55.7, 111.9, 128.3, 130.4, 130.7, 134.3, 158.1, 199.6;

m/z (ES⁺) 235 ([M-Na]⁺, 100%);

### {6-[5-(2-chloro-ethyl)-2-hydroxy-phenyl]-6-oxo-hexyl}-carbamic acid 9H-fluoren-9-ylmethyl ester: Route to Compound F1

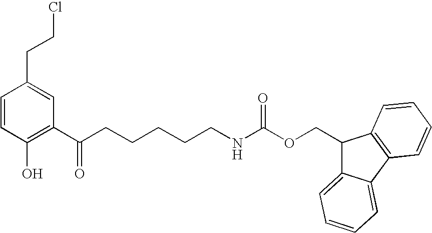

To a solution of Fmoc-ε-Ahx-OH (2.00 g, 5.66 mmol) in dry dichloromethane (10 ml) was added dropwise thionyl chloride (2.5 ml, 34.2 mmol). The solution was heated at 40° C. for 15 min. The solvent and excess of thionyl chloride were evaporated under vacuo and the remaining white solid was used without further purification. To the acyl chloride was added nitrobenzene* (20 ml) followed by 4-hydroxyphenethyl chloride (0.920 g, 5.7 mmol) in nitrobenzene (10 ml). The solution was cooled down to 0° C. and aluminium chloride (2.6 g, 19.5 mmol) was added portionwise. The solution was then heated at 57° C. for 16 h. Water (20 ml) was then added and the mixture extracted with diethyl ether (2×50 ml). The combined organic phases were dried over MgSO₄ and the solvent evaporated under vacuo. The crude mixture was purified using flash silica gel chromatography (chloroform then chloroform/MeOH 5%)** to give the titled compound (0.90 g, 34%)

¹H NMR (400 MHz; CDCl₃) δ 12.27 (1H, s, OH), 7.76 (2H, d, J 7.5 Hz, H—Fmoc), 7.58 (3H, m, H-Fmoc, H—Ar), 7.39 (2H, t, J 7.4 Hz, H-Fmoc), 7.32 (3H, m, H-Fmoc, H—Ar), 6.94 (1H, d, J 8.5 Hz, H—Ar o-OH), 4.83 (1H, t broad, NH), 4.40 (2H, d, J 6.8 Hz, COOCH₂CH), 4.21 (1H, t, J 6.6 Hz, COOCH₂CH), 3.69 (2H, t, J 7.0 Hz, CH₂Cl), 3.2 (2H, q, J 6.4 Hz, CH₂NH), 3.0 (4H, m, CH₂CH₂Cl, CH₂OAr), 1.76 (2H, m, CH₂CH₂), 1.55 (2H, m, CH₂CH₂), 1.44 (2H, m, CH₂CH₂)

¹³C NMR (100 MHz; CDCl₃) δ 207.1 (CO), 161.4 (Ar C—H), 156.4 (NHCOO), 143.9 (C-q), 141.3 (C-q), 136.7 (C-q), 130.0 (Ar—CH), 128.4 (Ar—CH), 127.6 (Ar—CH), 126.9 (Ar C—H), 124.9 (Ar C—H), 119.9 (Ar C—H), 119.0 (Ar C—H), 118.8 (Ar C—H), 66.5 (COOCH₂), 53.4 (CH₁₂CO), 47.2 (COOCH₂CH), 45.0 (CH₂Cl), 40.7 (CH₂CH₂Cl), 38.0 (CH₂NHCOO), 29.8 (CH₂), 26.2 (CH₂), 23.8 (CH₂).

m/z FAB 514 [(M+Na), 100%]

### Notes:

*Carbon tetrachloride was previously used as solvent following some previous literature procedures, but unfortunately the only product isolated was:

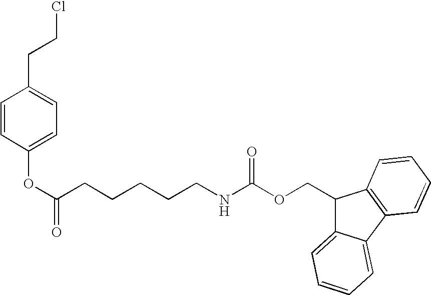

** Chloroform was first used to remove the nitrobenzene, then, by increasing the polarity (methanol/chlorofom), the product can be eluted. However, there was a small impurity which co-ran with the product and therefore the amount of methanol used was 2-5% in chloroform.

### {6-[5-(2-Chloroethyl)-2-hydroxyphenyl]-6-hydroxylhexyl}-carbamic acid 9H-fluoren-9-ylmethyl ester

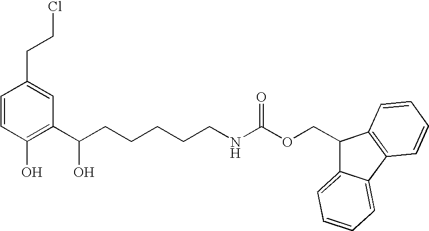

To a solution of the Fmoc ketone (0.200 g, 0.40 mmol) in dry methanol (8 ml)* was added NaBH₄ (20 mg, 0.52 mmol). The solution was heated at reflux for 16 h and then solvent removed in vacuo. The residue was redissolved in chloroform and washed with water (10 ml) and brine (10 ml). The organic phase was dried over MgSO₄ and the solvent evaporated under vacuo. The residue was then purified using flash column chromatography (chloroform/MeOH, 7/1)** to give the titled compound (0.100 g, 50%)

¹H NMR (400 MHz; CDCl₃) δ 7.95 (1H, s broad, OH), 7.75 (2H, d, J 7.5 Hz, H-ArFmoc), 7.57 (2H, d, J 7.5 Hz, H—ArFmoc), 7.39 (2H, t, J 7.4 Hz, H—ArFmoc), 7.30 (2H, t, J 7.4 Hz, H—ArFmoc), 6.98 (1H, J 8.2 Hz, H—Ar), 6.80 (1H, d, J 8.2 Hz, H—Ar), 6.78 (1H, s, H—Ar), 4.80-4.77 (2H, m, NH, CHOH), 4.40 (2H, d, J 6.7 Hz, COOCH₂CH), 4.20 (1H, t, COOCH₂CH), 3.64 (2H, t, J 8.0 Hz, CH₂Cl), 3.10 (2H, m, CH₂NH), 2.94 (2H, t, J 7.4 Hz, CH₂CH₂Cl), 1.76 (1H, m, CH₂CH₂), 1.70 (1H, m, CH₂CH₂), 1.48-1.35 (6H, m, CH₂CH₂)

¹³C NMR (100 MHz; CDCl₃) δ 156.6 (NHCOO), 154.4 (Ar C-1), 143.9 (C q), 141.3 (C q), 129.1 (C q), 129.0 (Ar C-3), 127.6 (Ar C-5), 127.5 (Ar Fmoc C-3), 127.0 (Ar Fmoc C-4), 124.9 (Ar Fmoc C-5), 119.9 (Ar Fmoc C-2), 117.3 (Ar C-2), 75.6 (CHOH), 66.5 (COOCH₂), 47.2 (COOCH₂CH), 45.3 (CH₂Cl), 40.6 (CH₂CH₂Cl), 38.3 (CH₂NHCOO), 37.0 (CH₂CHOH), 29.8 (CH₂), 25.9 (CH₂), 24.9 (CH₂)

### 2-(6-Amino-1-hydroxyhexyl)-4-(2-chloroethyl)-phenol—Compound F1

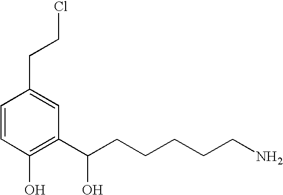

To a solution of compound the Fmoc alcohol (0.36 mg, 0.073 mmol) in DMF (5 ml) was added piperidine (1 ml). The solution was stirred at room temperature for 20 min and the solvents were then evaporated under high vacuo. The solid was then dissolved in chloroform and washed thoroughly with hexane. Compound F1 was finally obtained in 75% yield*.

¹H NMR (500 MHz; CD₃OD) δ 7.13 (1H, s, H-5), 6.94 (1H, d, J 8.1 Hz), 6.67 (1H, d, J 8.1 Hz), 4.94 (1H, m, CHOH), 3.66 (2H, t, J 7.1 Hz, CH₂Cl), 2.93 (2H, t, J 7.3 Hz, CH₂CH₂Cl), 2.84 (2H, t, J 7.5 Hz, CH₂NH₂), 1.72 (2H, m, CH₂CHOH), 1.60 (2H, m, CH₂), 1.5-1.4 (4H, m, CH₂CH₂)

¹³C NMR (125 MHz; CD₃OD) δ 154.3, 132.1, 130.4, 129.2, 128.0, 116.2, 70.4 (CHOH), 46.3 (CH₂Cl), 40.9 (CH₂NH₂), 39.7 (CH₂CH₂Cl), 38.5 (CH₂), 29.1 (CH₂), 27.4 (CH₂), 26.5 (CH₂)

m/z ES(+) 272.2 [M+H, 100%]

(3-Bromopropyl)phenol—Compound K1 (JW4) (C. J. Cooksey, P. J. Garratt, E. J. Land, S. Pavel, C. A. Ramsden, P. A. Riley, N. P. M. Smit, J. Biol. Chem., 1997, 272, 26226)

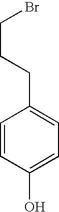

A solution of 3-(4-hydroxyphenyl)-1-propanol (2.50 g, 16.5 mmol), sulfuric acid (1 ml) and aqueous hydrobromic acid (48%, 15 ml) was heated at reflux for 6 h, After cooling to rt, the reaction mixture was neutralised with saturated sodium hydrogencarbonate solution, and then washed with ethyl acetate (3×60 ml). The combined organic layers were washed with brine (100 ml), dried over magnesium sulfate, and then concentrated in vacuo. Purification by flash chromatography on silica (dichloromethane) afforded the titled compound as a pale yellow solid (2.70 g, 78%).

¹H NMR (300 MHz; CDCl₃) [symbol] 2.15 (2H, tt, J 6.6, 6.6 Hz), 2.78 (2H, t, J 6.6 Hz), 3.38 (2H, t, J 6.6 Hz), 6.79 (2H, d, J 9.0 Hz, Ph-H), 7.06 (2H, d, J 9.0 Hz, Ph-H);

¹³C NMR (75.5 MHz; CDCl₃) 33.0, 33.2 and 34.4, 115.3, 129.7, 132.8, 153.8.

m/z (−ES) 215 (100, [M−H]⁻).

### 2-(4-Methoxyphenyl)-N,N-dimethylethanamine—Compound O1 (JW8) (Y. Sato, H. Sakakibara, J. Orgaitometallic Chem. 1979, 166, 303.)

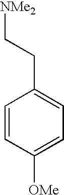

A solution of 3-(4-methoxyphenyl)-1-ethanol (0.30 g, 1.39 mmol) and dimethylamine solution (2.0 M in THF, 2 mL, 4.00 mmol) was stirred in a sealed-tube at rt for 18 hr. The reaction was concentrated in vacuo. Purification by flash chromatography on silica (10% methanol in dichloromethane) afforded the titled compound as a white solid (155 mg, 55%).

¹H NMR (300 MHz; CDCl₃) 2.67 (6H, s), 2.99 (4H, m), 3.83 (3H, s), 6.75 (2H, d, J 8.6 Hz, Ph-H), 7.08 (2H, d, J 8.6 Hz, Ph-H);

¹³C NMR (75.5 MHz; CDCl₃) 30.7, 43.6, 55.3, 59.7, 114.2, 128.6, 129.7, 158.6;

m/z (+ES) 180 (100, MH⁺).

### 4-(3-(Methylamino)propyl)phenol—Compound Q1/K2 (JW 11)

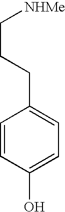

To a Solution of 4-(3-Bromopropyl)Phenol (2.00 G, 10.1 Mmol) and Tert-Butyldimethylsilyl chloride (1.68 g, 11.1 mmol) in THF (40 ml) was added slowly imidazole (1.88 g, 27.6 mmol). The reaction mixture was stirred for 4 h, filtered, and then concentrated in vacuo. The concentrated filtrate was re-dissolved in ethyl acetate (60 ml) and washed with water (60 ml), saturated sodium hydrogencarbonate solution (60 ml), and brine (60 ml). The organic layer was dried over magnesium sulfate and then concentrated in vacuo. Purification by flash chromatography on silica (5% dichloromethane in hexane) afforded the silylated phenol as a colourless oil (2.95 g, 89%).

¹H NMR (300 MHz; CDCl₃) δ−0.01 (6H, s), 0.98 (9H, s), 2.12 (2H, tt, J 6.6, 6.6 Hz), 2.70 (2H, t, J 6.6 Hz), 3.38 (2H, t, J 6.6 Hz), 6.76 (2H, d, J 8.5 Hz, Ph-H), 7.04 (2H, d, J 8.5 Hz, Ph-H);

¹³C NMR (75.5 MHz; CDCl₃) [symbol] −4.4, 18.2, 25.7, 33.1 and 34.4, 120.0, 129.4, 133.1, 154.0;

m/z (+ES) 353 (40, [M+Na]⁺), 360 (100).

A solution of the silylated intermediate (0.50 g, 1.52 mmol) and methylamine solution (33% in ethanol, 1 ml) was stirred in a sealed-tube at rt for 18 h. The reaction mixture was concentrated in vacuo, and then re-dissolved in a solution of concentrated HCl/water/methanol (1:1:5, 21 ml). The resulting mixture was stirred for a further 72 h, then neutralized with saturated sodium hydrogencarbonate solution and extracted with ethyl acetate (3×30 ml). The combined organic layer was washed with brine (50 ml), dried over sodium sulfate, and then concentrated in vacuo. Purification by flash chromatography on silica (aqueous ammonia solution/methanol/dichloromethane, 5:20:75) afforded the compound Q1 as a pale yellow solid (56 mg, 22%).

¹H NMR (300 MHz; CDCl₃) [symbol] 1.83 (2H, tt, J 7.4, 7.5 Hz), 2.43 (3H, s), 2.54 (2H, t, J 7.4 Hz, CH₂), 2.63 (2H, t, J 7.5 Hz, CH₂), 6.76 (2H, d, J 8.5 Hz, Ph-H), 6.93 (2H, d, J 8.5 Hz, Ph-H);

¹³C NMR (75.5 MHz; CDCl₃) [symbol] 30.7, 32.5, 35.6, 50.9, 115.7, 129.3, 132.1, 155.3;

m/z (+ES) 165 (100, MH⁺).

4-(2-(Dimethylamino)ethyl)phenol—compound T1/L2 (JW32) (H. Voswinckel, Ber. 1912, 45 1004)

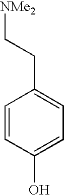

A solution of 4-methoxyphenethyl bromide (0.20 g, 0.93 mmol) and dimethylamine (2.0 M in THF, 2 ml) was stirred in a sealed-tube at rt for 18 h. The reaction mixture was concentrated in vacuo and re-dissolved in dichloromethane (2 ml). After cooling to 0° C., boron tribromide (1.0 M in hexane, 1.00 ml) was added dropwise, and the solution was stirred at this temperature for 10 min. Water (20 ml) was added dropwise and the mixture was stirred for further 30 min. After warming to rt, the reaction mixture was extracted with dichloromethane (3×20 ml). The combined organic layers were washed with brine (30 ml), dried over sodium sulfate, and then concentrated in vacuo. Purification by flash chromatography on silica (aqueous ammonia solution/methanol/dichloromethane, 5:20:75) afforded the titled compound as a white solid (61 mg, 40%).

¹H NMR (400 MHz; CD₄OD) [symbol] 2.31 (6H, s), 2.54 (2H, m, CH₂), 2.65 (2H, m, CH₂), 6.67 (2H, d, J 8.4 Hz, Ph-H), 6.99 (2H, d, J 8.4 Hz, Ph-H);

¹³C NMR (100 MHz; CD₄OD) [symbol] 34.5, 46.1, 63.5, 117.2, 131.4 and 132.3, 157.8;

m/z (+ES) 166 (100, MH⁺).

### 2-(4-{2-[2-(2-Methoxyethoxy)-ethoxy]-ethoxy}-phenyl)-ethanol—Compound X1 (JMB1)

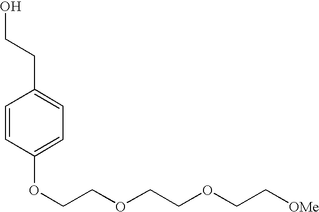

To a solution of triethylene glycol monomethyl ether (0.50 ml, 3.1 mmol) in dichloromethane (5 ml) was added p-toluenesulfonyl chloride (715 mg, 3.75 mmol) and triethylamine (0.52 ml, 3.8 mmol) and the reaction mixture was stirred at rt, for 24 h under nitrogen. The solution was washed with water (3×5 ml) and the organic layer separated and dried over magnesium sulfate. Dichloromethane was removed in vacuo and the crude product was purified by silica column chromatography (eluting with ethyl acetate/hexane, 2:1) to afford toluene-4-sulfonic acid 2-[2-(2-methoxy-ethoxy)-ethoxy]-ethyl ester as a clear oil (870 mg, 88%) which was used in the next coupling step.

δ_{H} (300 MHz; CDCl₃) 2.44 (3H, s, CH₃Ar), 3.37 (3H, s, CH₃OCH₂), 3.53 (2H, m, PEG), 3.61 (8H, m, PEG), 3.68 (3H, t, J 4.8 Hz, CH₂CH₂OTs), 4.16 (3H, t, J 4.9 Hz, CH₂OTs), 7.34 (2H, d, J 8.1 Hz), 7.80 (2H, d, J 8.3 Hz);

δ_{C} (75 MHz; CDCl₃) 21.4, 58.9, 68.5, 69.0, 70.4, 70.6, 71.7, 127.8, 129.6, 132.9, 144.6;

m/z (ES+) 341 ([M+Na]⁺, C₁₄H₂₂O₆S, 100%), 319 ([M+H]⁺, 25%).

To a solution of 2-(4-hydroxyphenyl)ethanol (100 mg, 7.24×10⁻¹ mmol) in THF (5 ml) was added sodium hydride (60% in mineral oil, 48 mg, 0.72 mmol), with stirring at rt under nitrogen. The tosylated PEG above (230 mg, 7.24×10⁻¹ mmol) was added and the solution was heated at reflux for 16 h. The solution was cooled to rt and water (5 ml, 1 ml min⁻¹) was added dropwise with stirring. The solution was extracted with chloroform (10 ml) and the organic layer was separated and washed with water (3×5 ml). The organic layer was dried over magnesium sulfate and solvent removed in vacuo to afford the title compound as an orange oil (146 mg, 67%).

δ_{H} (300 MHz; CDCl₃) 2.80 (2H, t, J 6.5 Hz, CH₂CH₂OH), 3.37 (3H, s, CH₃OCH₂), (3.55, 2H, m, PEG), 3.64-3.74 (10H, m, PEG), 3.85 (2H, t, J 5.2 Hz, CH₂OH), 4.11 (2H, t, J 4.7 Hz, CH₂OAr), 6.83 (2H, d, J 8.6 Hz), 7.13 (2H, d, J 8.6 Hz);

δ_{C} (75 MHz; CDCl₃) 38.3 (CH₂CH₂OH), 59.0, 63.8 (CH₂OH), 67.5, 69.8, 70.6, 70.7, 70.8, 72.0, 114.8, 129.9, 130.6, 157.5;

m/z (ES+) 307 ([M+Na]⁺, C₁₅H₂₄O₅, 100%), 285 ([M+H]⁺, 55%).

### Compound Y1 (JMB2)

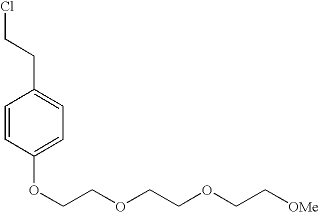

To a solution of compound X1 (50 mg, 0.18 mmol) in dichloromethane (5 ml) was added dimethylformamide (˜0.001 ml, cat.) and thionyl chloride (0.03 ml, 0.2 mmol) and the reaction mixture was stirred at rt for 16 h under nitrogen. The solution was washed with water (3×5 ml) and the organic layer was dried over magnesium sulfate. Dichloromethane was removed in vacuo and the crude product was separated by silica column chromatography (eluting with chloroform/methanol, 95:5) to afford Y1 as a yellow oil (39 mg, 72%).

δ_{H} (300 MHz; CDCl₃) 3.00 (2H, t, J 7.4 Hz, CH₂CH₂Cl), 3.38 (3H, s, CH₃OCH₂), 3.56 (2H, m, PEG). 3.65-3.74 (8H, m, CH₂Cl and PEG), 3.85 (2H, t, J 4.8 Hz, CH₂CH₂OAr), 4.11 (2H, t, J 4.7 Hz, CH₂OAr), 7.87 (2H, d, J 8.6 Hz), 7.13 (2H, d, J 8.6 Hz);

δ_{C} (75 MHz; CDCl₃) 38.4 (CH₂CH₂Cl), 45.2 (CH₂Cl), 59.0, 67.4, 69.8, 70.6, 70.7, 70.8, 71.9, 114.7, 129.8, 130.4, 157.8;

m/z (ES+) 325 ([M+Na]⁺, C₁₅H₂₃O₄Cl, 100%).

### 4-(2-Chloroethyl)-2-(5-(2-chloroethyl)-2-{2-[2-(2-methoxyethoxy)-ethoxy]-ethoxy}-benzyl)-phenol—Compound A2/P2 (JMB4)

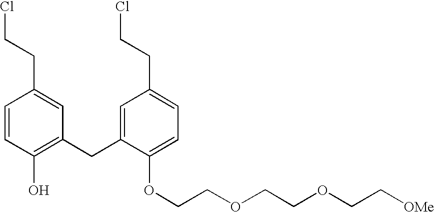

To a solution of CHLORINATED DIMER (105 mg, 3.23×10⁻¹ mmol) in dimethylformamide (5 ml) was added the tosylated PEG (see X1) (103 mg, 3.23×10⁻¹ mmol), potassium carbonate (45 mg, 0.32 mmol) and 18-Crown-6 (86 mg, 0.32 mmol) and the solution was stirred at rt for 16 h under nitrogen. Water (5 ml) was added and the solution was extracted with ethyl acetate (3×5 ml). The combined organic layers were dried over magnesium sulfate and the solvent was removed in vacuo. Separation of the crude product by silica column chromatography (eluting with chloroform/methanol, 95:5) afforded the title compound as a clear oil (4 mg, 0.009 mmol, 4%).

m/z (ES+) 493 ([M+Na]⁺, C₂₄H₃₂O₅Cl₂, 50%), 187 (100%).

### 2-Bromo-4-(2-chloroethyl)-phenol—Compound C2 (RB2B)

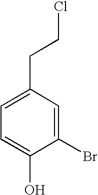

Bromine (0.20 ml, 3.90 mmol) was added to a stirring solution of 4-(2-chloroethyl)-phenol (600 mg, 3.83 mmol) in chloroform (20 ml) at 0° C. and the reaction mixture was stirred for 3.5 hours. The reaction mixture was quenched with saturated sodium hydrogen carbonate solution (20 ml). The organic layer was separated and washed with water (3×20 ml) and brine (20 ml), dried (MgSO₄), filtered and evaporated under reduced pressure to give the phenol as an orange oil (804 mg, 89%). R_{F} 0.21 (4:1 hexane:ethyl acetate);

νₘₐₓ/cm⁻¹ (film) 3501, 1607, 1497, 1123, 914 and 822;

δ_{H} (300 MHz; CDCl₃) 7.40 (1H, s, Ar), 7.08 (2H, d, J 8.3 Hz, Ar), 6.97 (1H, d, J 8.3 Hz, Ar), 5.56 (1H, s, OH), 3.67 (2H, t, J 7.2 Hz, CH₂Cl), 2.98 (2H, t, J 7.2 Hz, CH₂Ar);

δ_{C}(75 MHz; CDCl₃) 151.2, 132.1, 131.8, 116.1, 110.2, 44.9 and 37.9;

m/z (CI+) found M⁺234.9530; C₈H₈BrClO requires M⁺234.9525.

2-(4-Methoxyphenyl)-N,N,N-trimethylethanaminium bromide—Compound G2 (JW29) (J. R. I. Eubanks, L. B. Sims, A. Pry, J. Am. Chem. Soc. 1991, 113, 8821)

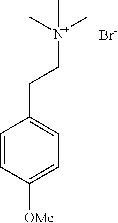

A solution of 4-methoxyphenethyl bromide (0.20 g, 0.93 mmol) and aqueous trimethylamine (45%, 0.22 ml) in THF (0.5 ml) was stirred in a sealed-tube at 50° C. for 18 h. After cooling to rt, the resulting mixture was neutralised with saturated sodium hydrogencarbonate solution and then extracted with ethyl acetate (3×10 ml). The combined organic layers were washed with brine (20 ml), dried over magnesium sulfate, and then concentrated in vacuo. Purification by flash chromatography on silica (10% methanol in dichloromethane) afforded the titled compound as a pale yellow solid (0.15 g, 63%).

δ_{H}(300 MHz; CD₄OD) 3.08 (2H, m), 3.24 (9H, s), 3.56 (2H, m), 3.76 (3H, s), 6.89 (2H, d, J 8.6 Hz, Ph-H), 7.26 (2H, d, J 8.6 Hz, Ph-1);

δ_{C} (100 MHz; CD₄OD) 29.4, 53.8, 55.8, 68.6, 115.4, 129.5 and 131.2, 160.4;

m/z (+ES) 194 (50, MH⁺), 135 (100, [M-NMe₃]⁺).

4-(2-(Methylamino)ethyl)phenol—Compound H2 (JW32) (V. N. Bulavka, A. N. Shchavlinskii, O. N. Tolkachev, Proc. ECSOC-3 and ECSOC-4 Sep. 1-30, 1999 and 2000, 142-146)

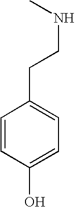

A solution of 4-methoxyphenethyl bromide (0.20 g, 0.93 mmol) and methylamine (33% in ethanol, 2 ml) was stirred in a sealed-tube at rt for 18 h. The reaction mixture was evaporated in vacuo and then re-dissolved in dichloromethane (2 ml). After cooling to 0° C., boron tribromide (1.0 M in hexane, 1.00 ml) was added dropwise, and the solution was stirred at this temperature for 10 min. Water (20 ml) was added dropwise and the mixture was stirred for a further 30 min. After warming to rt, the reaction mixture was extracted with dichloromethane (3×20 ml). The combined organic layers were washed with brine (30 ml), dried over sodium sulfate, and then concentrated in vacuo. Purification by flash chromatography on silica (aqueous ammonia solution/methanol/dichloromethane, 5:20:75) afforded the titled compound as a white solid (54 mg, 36%).

¹H NMR (400 MHz; CD₄OD) 2.42 (3H, s), 2.71-2.84 (4H, m), 2.65 (2H, m, CH₂), 6.74 (2H, d, J 8.5 Hz, Ph-H), 7.05 (2H, d, J 8.5 Hz, Ph-H);

¹³C NMR (100 MHz; CD₄OD) 36.2 and 36.6, 55.0, 117.3, 131.5 and 132.0, 157.9;

m/z (+ES) 152 (40, MH⁺), 120 (100, [M-NMe₃]⁺).

1-(3-Bromopropyl)-4-methoxybenzene—Compound I2 (JW31) (A. P. Tamiz, E. R. Whittemore, R. M. Woodward, R. B. Upasani, J. F. W. Keana, Biorg. Med. Chem. Lett. 1999, 9, 1619.)

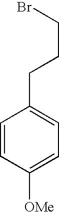

A solution of 3-(4-methoxyphenyl)-1-propanol (2.72 g, 16.5 mmol), sulfuric acid (1 ml) and aqueous hydrobromic acid (48%, 15 ml) was stirred at reflux for 6 h, After cooling to rt, the reaction mixture was neutralised with saturated sodium hydrogencarbonate solution, and then washed with ethyl acetate (3×60 ml). The combined organic layers were washed with brine (100 ml), dried over magnesium sulfate, and then concentrated in vacuo. Purification by flash chromatography on silica (dichloromethane) afforded the titled compound as a colourless oil (1.58 g, 42%).

¹H NMR (300 MHz; CDCl₃) 2.15 (3H, m), 2.71 (4H, t, J 7.5 Hz), 3.20 (2H, t, J 6.8 Hz), 3.80 (3H, s), 6.85 (2H, d, J 8.6 Hz, Ph-H), 7.02 (2H, d, J 8.6 Hz, Ph-H);

¹³C NMR (75.5 MHz; CDCl₃) 33.1, 34.4 and 35.2, 55.3, 114.0, 129.5, 132.6, 158.1;

m/z (+ES) 230 (30, MH⁺), 135 (100, [M-CH₂Br]⁺).

### Acetic acid 4-(2-chloroethyl)-phenyl ester—Compound M2 (RG26)

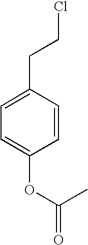

Acetyl chloride (0.24 ml, 3.38 mmol) was added to a stirring solution of 4-(2-chloro-ethyl)-phenol (261 mg, 1.67 mmol), pyridine (0.68 ml, 8.41 mmol) and 4-dimethylaminopyridine (20 mg, 0.16 mmol) in dichloromethane (6 ml) at 0° C. and the reaction mixture was allowed to warm slowly to room temperature and stirred for 17 h. The reaction mixture was quenched with water (8 ml). The organic layer was separated and washed with saturated sodium hydrogen carbonate solution (10 ml), water (3×10 ml), brine (10 ml), dried (MgSO₄), filtered and evaporated under reduced pressure to give the phenyl ester as a yellow oil (270 mg, 82%).

R_{F} 0.40 (4:1 hexane:ethyl acetate);

νₘₐₓ/cm⁻¹ (film) 2959, 1767, 1605, 1508, 1167, 1018 and 847;

δ_{H} (300 MHz; CDCl₃) 7.23 (2H, d, J 8.5 Hz, Ar), 7.04 (2H, d, J 8.5 Hz, Ar), 3.70 (2H, t, J 7.4 Hz, CH₂Cl), 3.06 (2H, t, J 7.4 Hz, CH₂Ar), 2.30 (1H, s. CH₃COO);

δ_{C} (75 MHz; CDCl₃) 169.5, 149.6, 135.7, 129.8, 121.7, 44.8, 38.6 and 21.1;

m/z (ES+) 221 (M+Na)⁺ (88%), (CI+) found M+199.0524; C₁₀H₁₁ClO₂ requires M⁺ 199.0520.

### Acetic acid 4-(2-acetoxy-ethyl)-phenyl ester—Compound N2

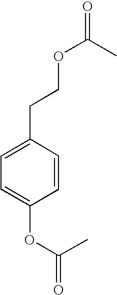

and

### Acetic acid 2-(4-hydroxyphenyl)-ethyl ester—Compound O2

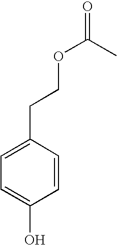

Pyridine (1.64 ml, 20.27 mmol) was added to a stirring solution of acetyl chloride (0.58 ml, 8.16 mmol), 2-(4-hydroxy-phenyl)-ethanol (510 mg, 3.69 mmol) and a catalytic amount of 4-dimethylaminopyridine in dichloromethane (13 ml) at 0° C. and the reaction mixture was allowed to warm slowly to room temperature and stirred for 20 h. The reaction mixture was quenched with water (20 ml). The organic layer was separated and washed with saturated sodium hydrogen carbonate solution (20 ml), 1M HCl (20 ml), water (3×20 ml), brine (20 ml), dried (MgSO₄), filtered and evaporated under reduced pressure to give the crude product, which was purified by flash chromatography, eluting with 4:1 hexane:ethyl acetate, to give the known diacetate (N2) (Procopiou, P. A., Baugh, S. P. D., Flack, S. S., Inglis, G. G. A. J. Org. Chem., 1998, 63, 2342-2347) as a yellow oil (391 mg, 48%).

R_{F} 0.43 (4:1 hexane:ethyl acetate);

νₘₐₓ/cm⁻¹ (film) 2959, 1740, 1506, 1367, 1167, 1018 and 851;

δ_{H} (300 MHz; CDCl₃) 7.22 (2H, d, J 8.5 Hz, Ar), 7.02 (2H, d, J 8.5 Hz, Ar), 4.26 (2H, t, J 7.0 Hz, CH₂OAc), 2.93 (2H, t, J 7.0 Hz, CH₂Ar), 2.29 (3H, s, CH₃COOCH₂);

δ_{C} (75 MHz; CDCl₃) 171.0, 169.6, 149.3, 135.4, 129.8, 121.6, 64.7, 34.5, 23.6, 21.1 and 21.0;

m/z (ES+) 245 (M+Na)⁺ (100%).

Also isolated from the above procedure was the known alkyl acetate (O2) (Shashidhar, M. S., Bhatt, M. V. J. Chem. Soc. Chem. Commun., 1987, 654.; Pedrochi-Fantoni, G., Servi, S. J. Chem. Soc. Perkin Trans. 1., 1992, 1029) as yellow needles (44 mg, 7 t). M.p. 54-57° C.;

R_{F} 0.27 (4:1 hexane:ethyl acetate);

νₘₐₓ/cm⁻¹ (nujol) 2979, 1644, 1620, 1485, 1148 and 1022;

δ_{H} (300 MHz; CDCl₃) 7.08 (2H, d, J 8.5 Hz, Ar), 6.75 (2H, d, J 8.4 Hz, Ar), 4.72 (1H, s, OH), 4.23 (2H, t, J 7.1 Hz, CH₂OAc), 2.86 (2H, t, J 7.1 Hz, CH₂Ar), 2.04 (3H, s, CH₃COO);

δ_{c} (75 MHz; CDCl₃) 171.5, 154.4, 130.0, 129.7, 115.4, 65.4, 34.2 and 21.0;

m/z (ES+) 203 (M+Na)⁺ (100%).

### Butyric acid 4-(2-chloro-ethyl)-phenyl ester—Compound R2 (JMB8)

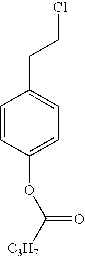

To a solution of 4-hydroxyphenethyl chloride (500 mg, 3.19 mmol) in dichloromethane (5 ml) was added butyryl chloride (0.40 ml, 3.8 mmol) and pyridine (0.31 ml, 3.8 mmol), with stirring, at 0° C. under nitrogen. The temperature was allowed to increase to rt and the reaction mixture was stirred for 16 h. The solution was washed with water (3×5 mL) and dried over magnesium sulfate. Dicholoromethane was removed in vacuo and the crude product was separated by silica column chromatography (eluting with hexane/ethyl acetate, 95:5) to afford the title compound as a clear oil (680 mg, 94%).

δ_{H} (300 MHz; CDCl₃) 1.04 (3H, t, J 7.4 Hz, CH₃CH₂), 1.76 (2H, sextet, J 7.4 Hz, CH₃CH₂CH₂), 2.53 (2H, t, J 7.4 Hz, CH₂CO₂Ar), 3.06 (2H, t, J 7.4 Hz, CH₂CH₂Cl), 3.70 (2H, t, J 7.4 Hz, CH₂C1), 7.03 (2H, d, J 8.5 Hz), 7.23 (2H, d, J 8.5 Hz);

δ_{C} (75 MHz; CDCl₃) 13.6, 18.5, 36.2, 38.5 (CH₂CH₂Cl), 44.8 (CH₂Cl), 121.7, 129.8, 135.5, 149.6, 172.2 (CH₂CO₂Ar);

m/z (ES+) 249 ([M+Na]⁺, C₁₂H₁₅O₂Cl, 100%).

### 2,2′-Methylenebis(4-(3-bromopropyl)phenol)—Compound S2 (JW35)

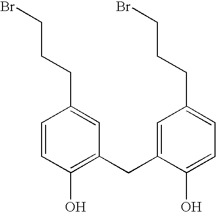

A solution of 4-(3-bromopropyl)phenol (0.30 g, 1.51 mmol), formaldehyde (0.12 ml, 1.51 mmol) and concentrated sulfuric acid (1 ml) in water (5 ml) was stirred at sealed-tube at 80° C. for 2 h. After cooling to rt, the reaction mixture was neutralised with saturated sodium hydrogencarbonate solution. The resulting mixture was extracted with ethyl acetate (3×20 ml). The combined organic solvent were washed with brine (40 ml), dried over magnesium sulfate, and then concentrated in vacuo. Purification by flash chromatography on silica (30% diethyl ether in hexane) afforded the titled compound as a white solid (72 mg, 23%).

¹H NMR (300 MHz; CD₄OD) δ 1.99 (4H, tt, J 6.6, 7.2 Hz), 2.57 (4H, t, J 7.2 Hz), 3.30 (4H, t, J 6.6 Hz), 3.83 (2H, s), 6.69-7.05 (6H, m, Ph-H);

¹³C NMR (75.5 MHz; CD₄OD) δ 31.0, 33.8, 34.1 and 35.9, 116.2, 128.2, 128.7, 131.7, 131.1, 154.0;

m/z (+ES) 465 (20, [M+Na]⁺), 304 (100, [M-2Br+Na]⁺;

m/z (+ES) 441 (100, [M−H]⁻).

### Bis(5-3-bromopropyl)-2-methoxyphenyl)methane—Compound T2 (JW37)

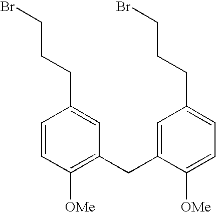

A solution of sodium hydride (60%, 22 mg, 0.54 mmol) in anhydrous THF (2 ml) was stirred in a sealed-tube at rt for 15 min. After heating to 50° C., S2 (0.12 g, 0.27 mmol) was added and the reaction mixture stirred at this temperature for 30 min. Iodomethane (34 μl, 0.54 mmol) was added and stirring was continued for 1 h. After cooling to rt, water (10 ml) was added and the mixture extracted with ethyl acetate (3×15 ml). The combined organic extracts were washed with brine (30 ml), dried over magnesium sulfate, and then concentrated in vacuo. Purification by flash chromatography on silica (10% dichloromethane in hexane) afforded the titled compound as a colourless oil (54 mg, 43%).

¹H NMR (300 MHz; CDCl₃) 2.07 (4H, m), 2.63 (4H, t, J 7.1 Hz), 3.35 (4H, t, J 6.6 Hz), 3.81 (6H, s), 3.94 (2H, s), 6.81-7.06 (6H, m, Ph-H);

¹³C NMR (75 MHz; CDCl₃) 30.0, 33.2, 34.5 and 35.2, 55.5, 110.3, 127.0, 129.1, 130.6, 132.1, 156.1.

m/z (+ES) 453 (50, [M+Na]⁺), 180 (100);

### Example 1

### Experiments Monitoring PKB Modulation

PKB is a protein downstream effector of PI3K, and becomes phosphorylated on (residues required for its activity) in response to the activation of PI3K. Natal Calf Serum (NCS) is a stimulator of PI3K and thus subsequently results in PKB activation. Therefore the positive control used in experiments is 10% serum and a negative control used is provided with no serum at all.

In a typical method, NIH3T3 cells were grown in media (GibcoBRL) containing 10% NCS to near confluency in six well plates. The cells were starved using 0.5% serum for 2-3 days. The media was then removed and replaced with serum free media for 15 minutes. Subsequently, 1% NCS was added to reaction wells, and 0%, 1% and 10% NCS to control wells. After 20 minutes incubation, the compound was added, and the wells incubated for a further 15 minutes. Media was removed and sample buffer was added and the cells lysed, boiled, centrifuged.

Samples were subjected to gel electrophoresis by 10% SDS-PAGE and then Western blotted on to PVDF membrane (Biorad) according to standard protocols. Western blots were probed using primary antibodies against PKB purchased from New England Biolabs and secondary antibodies of goat anti-rabbit IgG coupled to horseradish peroxidase (Amersham). The membranes were then developed using a freshly prepared ECL solution according to standard protocols.

The results for various concentrations of the compounds Q, B, D, E and F are shown in FIG. 2a, and corroborating results for compounds D and E are provided in FIG. 2b. The results indicate that compound E is an inhibitor of PKB, while compounds D and F are activators.

Western blotting of the phospho-Akt content was also monitored. The methodology used was the same as above. The results in FIG. 5 indicate that 9 compounds [A B C D E F I J Q] can activate PKB.

### Example 2

### Activation of PKB by c48/80 and cQ

Based on data that c48/80 (a condensation product of N-methyl-p-methoxyphenethylamine and formaldehyde and is a mixture of cationic amphiphiles of varying degrees of polymerisation) is an activator of PKB, our aim was to confirm the promising results obtained by Western Blotting with a different approach. We therefore tested the compound's effect on PKB S473 phosphorylation by immunofluorescence microscopy with a phospho-specific PKB antibody.

As c48/80 is a mixture of cationic amphiphiles of differing degrees of polymerisation, we aimed to find an activator from the purified synthetic single compounds. One of those analogues turned out to be an even more powerful tool to investigate PKB involving pathways. Therefore we concentrated our efforts on compound Q (cQ):

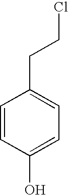

### Material and Methods

NIH 3T3 of Cos6 fibroblasts growing on PLL coated coverslips in DMEM containing 10% FCS were starved for 24 hours. Stimulation with c48/80 [10 μg/ml] was always carried out in the presence of 1% FCS for 10 min. Whereas cells growing in DMEM completely depleted of serum were used for experiments with cQ [15 μg/ml]. Where indicated, cells were pretreated with 100 μM LY294002 for 30 min or 500 nM RV001 for 15 min. After treatment cells were washed with PBS, fixed with 4% PFA and permeabilised with 0.25% Triton-X/PBS and washed extensively. After blocking in 1% BSA, cells were phalloidin stained and/or incubated with phospho-specific Ser473 PKB antibody (Cell Signalling) at 4° C. over night and a Fluorescein (FITC)—conjugated goat anti-mouse IgG (Jackson Immuno Research) at room temperature for 1 h. Nuclei were stained with Dapi. Coverslips were mounted on Mowiol containing slides and sealed before analysing on a Nikon Microscope.

c48/80 Induces the Phosphorylation of PKB on S473 Residue

In summary the imaging data are in agreement with Western Blot results and demonstrate that PKB phosphorylation occurs with doses from 3 μg/ml up to 10 μg/ml in NIH 3T3 and Cos 6 fibroblasts. c48/80 induced PKB activation was dependent on low amounts of serum, since treatment with c48/80 alone failed to induce PKB activation, as it was found before by Western Blotting. Consequently, c48/80 induced activation of PKB is sensitive to LY294002, a general PI3-kinase inhibitor, blocking phosphorylation induced by c48/80 in the presence of serum 1% serum or 10% serum alone (data not shown).

### cQ Induces the Phosphorylation of PKB but Acts as PKB Inhibitor in the Presence of Serum

In contrast to c48/80, cQ alone is sufficient to induce phosphorylation of PKB on S473 residue (FIG. 3c) without any serum present. Surprisingly, increasing concentrations of serum inhibit cQ induced PKB activation. As shown in FIG. 3b, after stimulation of starved cells with 10% FCS increased levels of phosphorylated PKB are detectable. In contrast, pretreatment with cQ completely abolished the activation of PKB by 10% FCS (FIG. 3d).

In order to investigate PI3-kinase dependency, studies with the PI3-kinase inhibitor LY294002 and the PTEN inhibitor RV001, which acts synergistically with growth factors upon PKB activation, were undertaken. The results demonstrate clearly that PI3-kinase activity is counteracting cQ induced PKB activation, and LY294002 treatment strongly increased cQ induced PKB phosphorylation (FIG. 3h). On the other hand RV001 treatment, which leads to increased PI(3,4,5)P₃ levels, generated by PI3-kinase, indirectly inhibits PKB activation after cQ challenge (FIG. 3g).

### Insulin-Stimulated Actin Remodeling is Inhibited by cQ

Preliminary data on phalloidin staining underline the results, that cQ is involved in a different pathway other than PKB acting as a downstream target of PI3-kinase upon activation of tyrosine-kinase and G-protein coupled receptors. cQ induces the loss of stress fibers in starved fibroblasts, like insulin does (FIG. 4). But unlike insulin, cQ is capable of reorganising the cytoskeleton independent of PI3-kinase (FIG. 4c). cQ treatment produces a disorganised cytoplasmic F-actin and an actin ring juxtaposed to the plasma membrane (FIG. 4b). It seems to counteract insulin-stimulated actin remodelling, as the decrease in the amount of F-actin stress fibers is not as pronounced in the presence of cQ where short cytoplasmic disorganised actin fibers remain.

### Example 3

### Activation of PKB by Compound C

50 μM of Compound C (cC):

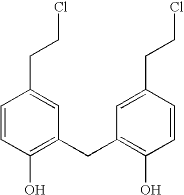

was found to activate Akt/PKB phosphorylation on S473 in starved cells and were inhibitory on Akt/PKB phosphorylation in Insulin stimulated cells. Consistent with those findings, PI3-kinase inhibition (Wortmannin or LY294002) led to an increase of phosphorylation at this site, whereas PTEN inhibition, and therefore increased PI(3,4,5)P3 levels inhibited the response in the presence of cC. As concentrations of 50 μM of compound C had cytotoxic effects on NIH3T3 fibroblasts (MTT assay), 1 μM concentrations were tested for its effects on Akt/PKB phosphorylation. The compound was still activatory on its own in starved cells. However its inhibitory effects on stimulated cells were not as strong. The methods used were as for Example 2.

### REFERENCES

Berge, S. M., L. D. Bighley, et al. (1977). “Pharmaceutical salts.” J Pharm Sci 66(1): 1-19.
Brazil, D. P., J. Park, et al. (2002). “PKB binding proteins. Getting in on the Akt.” Cell 111(3): 293-303.
Chang, F., J. T. Lee, et al. (2003). “Involvement of PI3K/Akt pathway in cell cycle progression, apoptosis, and neoplastic transformation: a target for cancer chemotherapy.” Leukemia 17(3): 590-603.
Evans, D. A. and P. A. Watson (1996). Tetrahedron Lett.: 3251-3254.
Glass, D. J. (2003). “Signalling pathways that mediate skeletal muscle hypertrophy and atrophy.” Nat Cell Biol 5(2): 87-90.
Hara, K., K. Yonezawa, et al. (1994). “1-Phosphatidylinositol 3-kinase activity is required for insulin-stimulated glucose transport but not for RAS activation in CHO cells.” Proc Natl Acad Sci USA 91(16): 7415-9.
Hill, M., J. Feng, et al. (2002). “Identification of a Plasma Membrane Raft-Associated PKB Ser473 Kinase Activity that Is Distinct from ILK and PDK1.”Curr Biol 12(14): 1251.
Hill, M. M. and B. A. Hemmings (2002). “Inhibition of protein kinase B/Akt. implications for cancer therapy.” Pharmacol Ther 93(2-3): 243-51.
Katsu, T., H. Ono, et al. (1984). Chem. Pharm. Bull 32: 4185-4188.
Kundra, V., J. A. Escobedo, et al. (1994). “Regulation of chemotaxis by the platelet-derived growth factor receptor-beta.” Nature 367(6462): 474-6.
Matsui, T., T. Nagoshi, et al. (2003). “Akt and PI 3-kinase signaling in cardiomyocyte hypertrophy and survival.” Cell Cycle 2(3): 220-3.
Newton, A. C. (2003). “Regulation of the ABC kinases by phosphorylation: protein kinase C as a paradigm.” Biochem J 370(Pt 2): 361-71.
Parekh, D. B., W. Ziegler, et al. (2000). “Multiple pathways control protein kinase C phosphorylation.” Embo J 19(4): 496-503.
Rodriguez-Viciana, P., P. H. Warne, et al. (1994). “Phosphatidylinositol-3-OH kinase as a direct target of Ras.” Nature 370(6490): 527-32.
Sabers, C. J., M. M. Martin, et al. (1995). “Isolation of a protein target of the FKBP12-rapamycin complex in mammalian cells.” J Biol Chem 270(2): 815-22.
Schu, P. V., K. Takegawa, et al. (1993). “Phosphatidylinositol 3-kinase encoded by yeast VPS34 gene essential for protein sorting.” Science 260(5104): 88-91.
Staal, S. P. (1987). “Molecular cloning of the akt oncogene and its human homologues AKT1 and AKT2: amplification of AKT1 in a primary human gastric adenocarcinoma.” Proc Natl Acad Sci USA 84(14): 5034-7.
Stephens, L., F. T. Cooke, et al. (1994). “Characterization of a phosphatidylinositol-specific phosphoinositide 3-kinase from mammalian cells.” Curr Biol 4(3): 203-14.
Tatton, W., D. Chen, et al. (2003). “Hypothesis for a common basis for neuroprotection in glaucoma and Alzheimer's disease: anti-apoptosis by alpha-2-adrenergic receptor activation.” Surv Opthalmol 48 Suppl 1: S25-37.
Valius, M. and A. Kazlauskas (1993). “Phospholipase C-gamma 1 and phosphatidylinositol 3 kinase are the downstream mediators of the PDGF receptor's mitogenic signal.” Cell 73(2): 321-34.
Vanhaesebroeck, B., S. J. Leevers, et al. (2001). “Synthesis and Function of 3-Phosphorylated Inositol Lipids.” Annu Rev Biochem 70: 535-602.
   Annu. Rev. Biochem. 2001. 70:535-602. SYNTHESIS AND FUNCTION OF 3-PHOSPHORYLATED INOSITOL LIPIDS
Varticovski, L., B. Druker, et al. (1989). “The colony stimulating factor-1 receptor associates with and activates phosphatidylinositol-3 kinase.” Nature 342(6250): 699-702.
   Colony stimulating factor-1 (CSF-1) is a lineage-specific growth factor
Vivanco, I. and C. L. Sawyers (2002). “The phosphatidylinositol 3-Kinase AKT pathway in human cancer.” Nat Rev Cancer 2(7): 489-501.
Filippa, N., Sable, C. L., Filloux, C., Hemmings, B., and Van Obberghen, E. (1999) Mol. Cell Biol. 19, 4989-5000
Gorin, Y., Kim, N. H., Feliers, D., Bhandari, B., Choudhury, G. G., and Abboud, H. E. (2001) FASEB J. 15, 1909-1920
Konishi, H., Matsuzaki, H., Tanaka, M., Takemura, Y., Kuroda, S., Ono, Y., and Kikkawa, U. (1997) FEBS Lett. 410, 493-498
Kanzaki, M. and Pessin, J. E. (2001) J. Biol. Chem. 276, 42436-42444

1-21. (canceled)

22. A method of modulating PKB activity in a subject in need thereof comprising administering to said subject a compound of the formula: 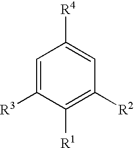
wherein R¹ is C₁₋₅ alkoxy, OCOC₁₋₃Alkyl, O(CH₂)₂O(CH₂)₂O(CH₂)₂OMe, O(CH₂)₂O(CH₂)₂O(CH₂)₂OH or OH;
R² is H, (CH₂)ₙOH, OCH₃, Hal or 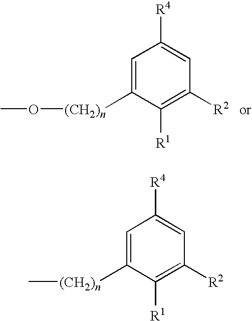
R³ is H or (CH₂)ₙOH; and
R⁴ is C₁₋₆ alkyl, optionally substituted by one or more of Hal, OH, COCH₃, NH₂, NHCH₃, NHMe, NMe₂, OCOCH₃, CO₂H or esters or amides thereof, where n is 1-5;
or a pharmaceutically acceptable salts thereof.

23. The method of claim 22 wherein PKB activity is inhibited.

24. The method of claim 22 wherein PKB activity is activated.

25. The method of claim 23 wherein the subject is suffering from cancer.

26. The method of claim 25 wherein the cancer is one in which a mutation of PTEN is implicated.

27. The method of claim 26 wherein the cancer is selected from the group consisting of ovarian, breast, prostrate, thyroid and pancreatic cancer.

28. The method of claim 23 wherein R¹ is Methoxy, R² and R³ are both H and R⁴ is (CH₂)₂COCH₃.

29. The method of claim 24 wherein the subject is suffering from a degenerative disease.

30. The method of claim 29 wherein the degenerative disease is selected from the group consisting of Alzheimers disease, stroke, infarction, hypoxia, skeletal muscle injury and type II diabetes.

31. The method of claim 24 wherein the compound is selected from the group consisting of:

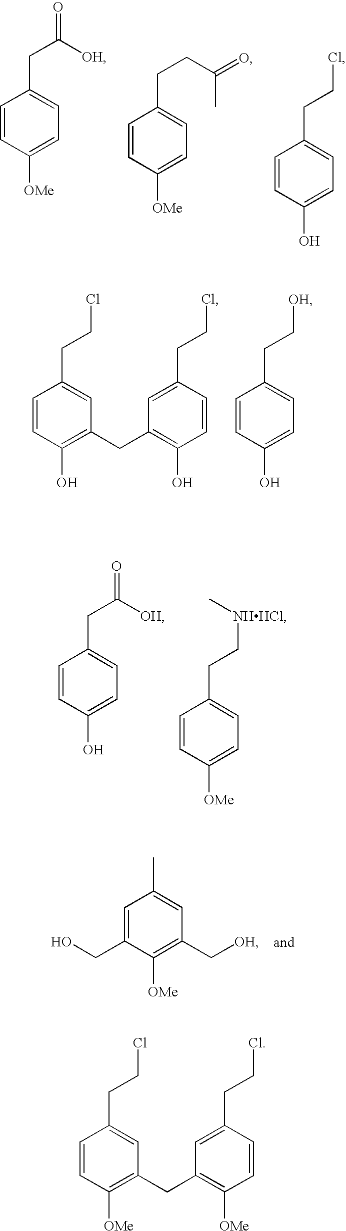

32. A pharmaceutical composition comprising a pharmaceutically acceptable carrier and a compound of the formula: 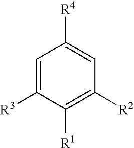
wherein R¹ is C₁₋₅ alkoxy, OCOC₁₋₃Alkyl, O(CH₂)₂O(CH₂)₂O(CH₂)₂OMe, O(CH₂)₂O(CH₂)₂O(CH₂)₂OH or OH;
R² is H, (CH₂)ₙOH, OCH₃, Hal or 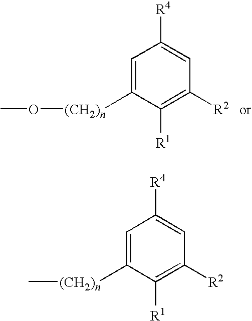
R³ is H or (CH₂)ₙOH; and
R⁴ is C₁₋₆ alkyl, optionally substituted by one or more of Hal, OH, COCH₃, NH₂, NHCH₃, NHMe, NMe₂, OCOCH₃, CO₂H or esters or amides thereof where n is 1-5;
or a pharmaceutically acceptable salts thereof.

33. A compound selected from the group consisting of:

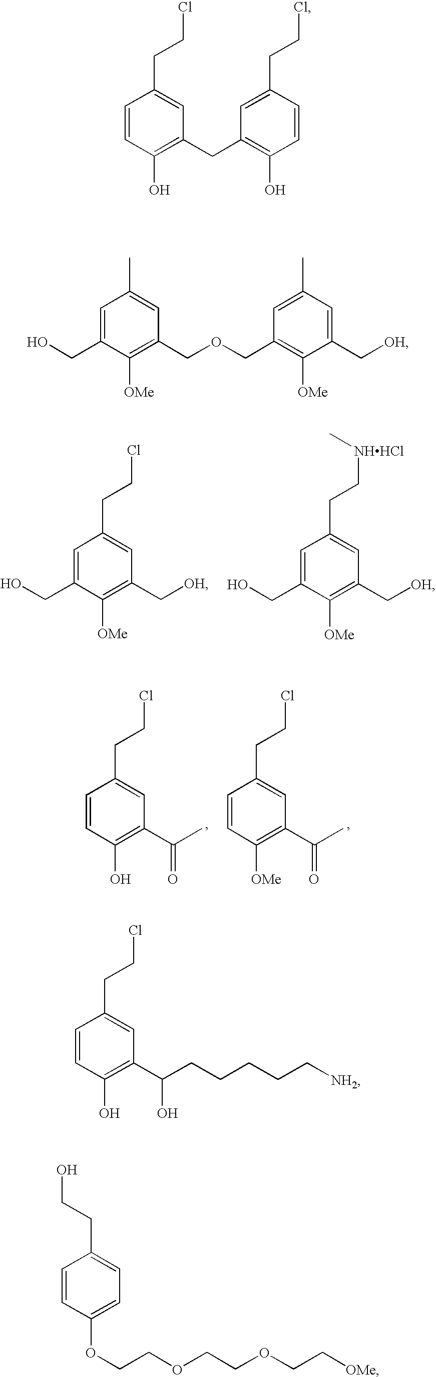

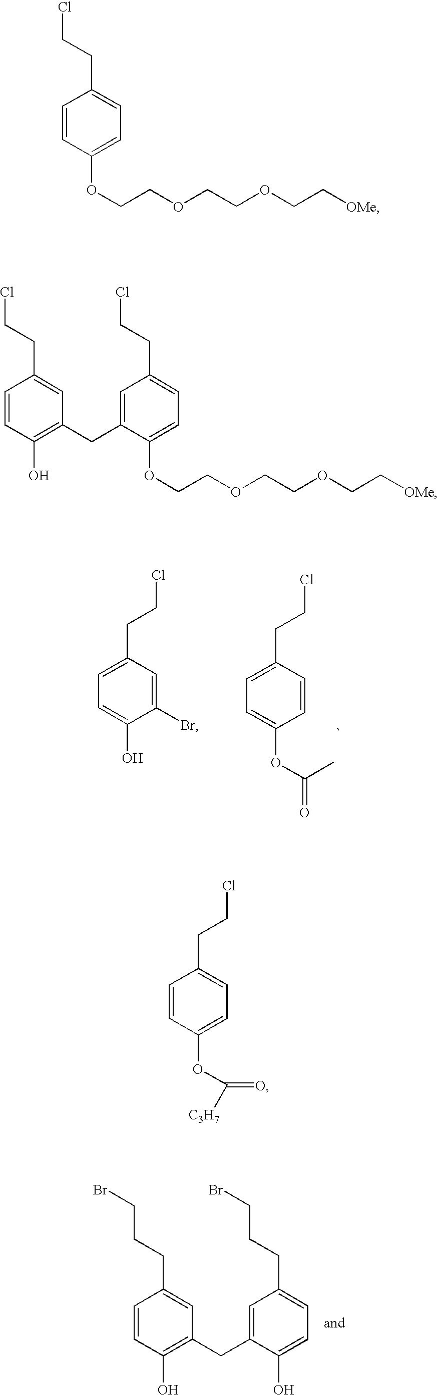

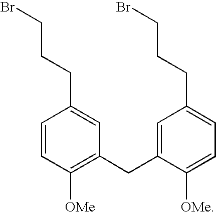

34. A compound of the formula: 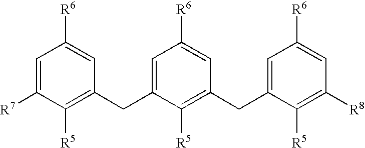
wherein R⁵ is C₁₋₅ alkoxy or OH;
R⁶ is C₁₋₅ alkyl, optionally substituted by Hal, NHCH₃, CO₂H or esters or amides thereof, and
R⁷ and R⁸ are independently (CH₂)_{q}OH where q is 2-5;
or a pharmaceutically acceptable salt thereof.

35. A method for modulating PKB activity in a subject in need thereof comprising administering to said subject a compound of claim 34.

36. The method of claim 35 wherein the PKB activity is inhibited.

37. The method of claim 36 wherein the subject in need of PKB modulation is suffering from cancer.

38. A pharmaceutical composition comprising a pharmaceutically acceptable carrier and a compound of claim 33.

39. A pharmaceutical composition comprising a pharmaceutically acceptable carrier and a compound of claim 34.
